(19) United States
(12) Patent Application Publication
(10) Pub. No.: US 2025/0312258 A1
(43) Pub. Date: Oct. 9, 2025

(54) 2,3-PYRROLIDINEDIONE CONJUGATES AND METHODS OF USING THEREOF

(71) Applicant: North Carolina State University, Raleigh, NC (US)

(72) Inventors: Joshua G. PIERCE, Raleigh, NC (US); Manuel Alejandro VALDÉS PEÑA, Raleigh, NC (US)

(21) Appl. No.: 18/855,125

(22) PCT Filed: Apr. 10, 2023
(86) PCT No.: PCT/US2023/018058

§ 371 Date: Oct. 8, 2024

Related U.S. Application Data
(60) Provisional application No. 63/328,804, filed on Apr. 8, 2022.

(51) Int. Cl. A61K 8/49; A61K 47/54; A61L 31/08; A61P 31/04

(52) U.S. Cl. CPC A61K 8/4913; A61K 47/545; A61L 31/08; A61P 31/04; A61L 2300/406

(57) ABSTRACT

Provided herein are compounds that can exhibit activity as biofilm modulating agents (e.g., activity as biofilm inhibitors and/or activity as biofilm dispersal agents). The compounds can exhibit potent activity against Gram positive biofilms. The compounds can also exhibit activity against Gram negative biofilms. In some cases, the compounds can exhibit both biofilm modulation properties and antimicrobial activity. Compositions comprising these compounds, as well as methods of using thereof, are also described. For example, the compounds described herein can be used in human and animal health (e.g., for the treatment of infection), agriculture, marine coatings, and other coating applications related to prevention of biofilm (e.g., dental, medical, etc.).

### CROSS-REFERENCE TO RELATED APPLICATIONS

This application claims benefit of U.S. Provisional Application No. 63/328,804, filed Apr. 8, 2022, which is hereby incorporated herein by reference in its entirety.

### BACKGROUND

The burden of disease caused by multi-drug resistant (MDR) bacterial pathogens is a global problem that is occurring among an alarming number of new pathogens each year. Not only is the problem exacerbated by the lack of new antimicrobials to treat these infections, but many of the most serious pathogens are now resistant to multiple classes of antimicrobials rendering treatment either extremely difficult or non-effective. With clinically relevant bacteria gaining resistance to common antibiotics at an alarming rate, the development of new treatments is a pressing need for public health and novel natural product scaffolds are fruitful starting points toward realizing this goal. Further, over the past decade there has been an increased awareness of the importance of biofilms in bacterial pathogenesis and there is great interest in potent and selective molecules that can modulate biofilms without bactericidal activity. Significantly, no FDA approved antibiotics or combinations thereof are successful in clearing biofilm implicated infections. The MDR situation is so critical that the World Health Organization has listed MDR bacterial pathogens as one of the top three threats to global health.

Accordingly, there is a critical need for compounds and compositions that can control biofilms, as well as improved methods for controlling biofilms.

### SUMMARY

Described herein are 2,3-pyrrolidinedione conjugates that exhibit potent antimicrobial activity, including against drug-resistant pathogens, and separately, anti-biofilm activity that is capable of eradicating robust biofilms. Importantly, these conjugates exhibit potent antimicrobial activity against drug-resistant bacterial strains, such as MRSA and VRSA, and a strong ability to impact bacterial biofilms (MBEC's 100-500× more potent than vancomycin) while retaining selectivity for bacterial cells over mammalian cells.

For example, provided herein are compounds defined by Formula I

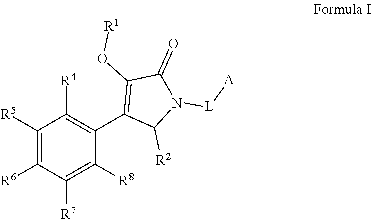

or a pharmaceutically acceptable salt or prodrug thereof, wherein
L is absent, or represents a bivalent linking group;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl.

In some embodiments of Formula I, L comprises from 2 to 30 carbon atoms.

In some embodiments, L can comprise an alkylene group, a cycloalkylene group, an alkylcycloalkylene group, an arylene group, an alkylarylene group, an oligo(alkyleneoxy) group, an oligo(alkyleneimine) group, or any combination thereof. Optionally, L can further comprises one or more functional groups, such as a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—), wherein R⁹ represents an alkyl group, an aryl group, or a heterocyclic group.

In some embodiments, L is not cleavable.

In some embodiments, L can comprise a positively charged moiety.

In some embodiments, the compound can be defined by Formula IA

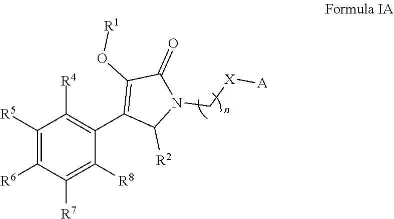

or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
n is an integer from 2 to 12;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

In some embodiments, the compound can be defined by Formula IB

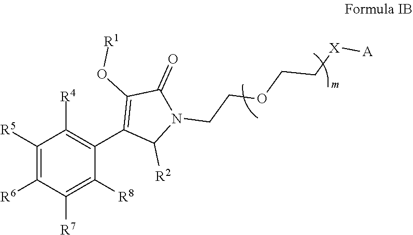

or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
m is an integer from 1 to 20;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

In some embodiments, the compound can be defined by Formula IC or Formula ID

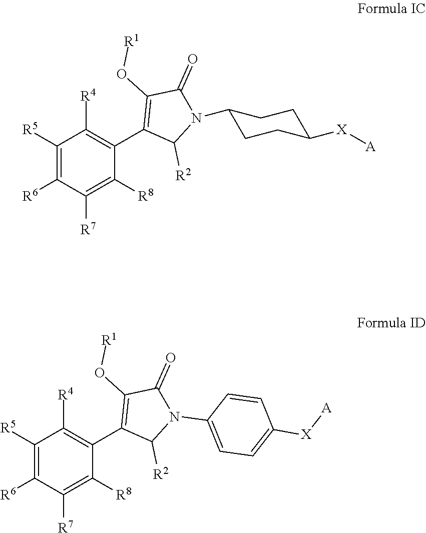

or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

In some embodiments of Formula I, IA, IB, IC, and ID, R¹ is hydrogen or a C₁-C₄ alkyl group optionally substituted with one or more substituents individually chosen from R⁹.

In some embodiments of Formula I, IA, IB, IC, and ID, R² is a C₁-C₄ alkyl group optionally substituted with one or more substituents individually chosen from R⁹. In other embodiments of Formula I, IA, IB, IC, and ID, R² is a alkylaryl group optionally substituted with one or more substituents individually chosen from R⁹, such as a benzyl or hydroxybenzyl group.

In some embodiments of Formula I, IA, IB, IC, and ID, at least one of R⁴, R⁵, R⁶, R⁷, and R⁸ is not hydrogen. In some cases, R⁴, R⁵, R⁶, R⁷, and R⁸ are all hydrogen.

In some embodiments of Formula I, IA, IB, IC, and ID, R⁶ is an electron withdrawing group. In certain cases, R⁶ can be a haloalkyl. In certain cases, R⁶ can be a perfluoroalkyl group (e.g., —CF₃).

In some embodiments of Formula I, IA, IB, IC, and ID, A can comprise an antibacterial agent. In some cases, the antibacterial agent can be an antibacterial agent that acts via an extracellular mechanism of action. For example, the antibacterial agent can target bacterial cell walls. Examples of such antibacterial agents include β-lactam antibiotics (e.g., penicillins, cephalosporins, monobactams, and carbapenems) glycopeptide antibiotics (e.g., teicoplanin, vancomycin, telavancin, dalbavancin, and oritavancin), and polypeptide antibiotics (e.g., bacitracin). In certain embodiments of Formula I, IA, IB, IC, and ID, A can comprise a glycopeptide antibiotic, such as vancomycin.

Also provided are compositions that can prevent, remove, and/or inhibit biofilms. Biofilm preventing, removing, or inhibiting compositions can comprise a carrier and an effective amount of a compound described herein to prevent, remove, and/or inhibit a biofilm. The composition can be, for example, a dentifrice composition (e.g., a toothpaste, mouthwash, chewing gum, dental floss, or dental cream) that promotes dental hygiene by preventing, reducing, inhibiting or removing a biofilm.

Also provided herein are pharmaceutical compositions that comprise a compound described herein in a pharmaceutically acceptable carrier. In some embodiments, pharmaceutical compositions can further include one or more additional active agents (e.g., one or more antibiotics).

The compounds described herein can also be disposed on or within a substrate to control biofilm formation on the substrate. Accordingly, also provided are medical devices that comprise a medical device substrate and an effective amount of a compound described herein either coating the substrate, or incorporated into the substrate. The effective amount of the compound can be an effective amount to prevent or inhibit growth of a biofilm on the medical device substrate. The medical device substrate can include, for example, a stent, fastener, port, catheter, scaffold, and/or graft.

Also provided herein are methods for controlling biofilm formation on a substrate. Methods for controlling biofilm formation on a substrate can comprise contacting the substrate with a compound described herein in an amount effective to inhibit biofilm formation. The biofilm can comprise Gram-positive bacteria or Gram-negative bacteria. In some embodiments, the biofilm can comprise Gram-positive bacteria (e.g., a bacteria of a genus Staphylococcus, such as Staphylococcus aureus).

Also provided herein are methods for treating chronic bacterial infections. Methods for treating a chronic bacterial infection in a subject in need thereof can comprise administering to said subject a compound described herein in an amount effective to inhibit, reduce, or remove a biofilm component of the chronic bacterial infection. The chronic bacterial infection can comprise, for example, a urinary tract infection, gastritis, a respiratory infection, cystitis, pyelonephritis, osteomyelitis, bacteremia, a skin infection, rosacea, acne, a chronic wound infection, infectious kidney stones, bacterial endocarditis, or a sinus infection.

Also provided are methods of treating subjects infected with a bacterium. Methods of treating a subject infected with a bacterium can comprise administering to the subject a therapeutically effective amount of a compound described herein. In some embodiments, the bacterium can comprise a Gram-positive bacterium. For example, the bacterium can include Staphylococcus aureus (methicillin sensitive), Staphylococcus aureus (methicillin resistant). Staphylococcus aureus (vancomycin resistant), Streptococcus pneumonia (penicillin sensitive), Streptococcus pneumonia (penicillin resistant), Staphylococcus epidermis (multiple drug resistant), Enterococcus faecalis (vancomycin sensitive), Enterococcus faecium (vancomycin resistant), and/or Haemophilus influenzae. In some embodiments, the bacterium can comprise a Gram-negative bacterium. For example, the bacterium can include Salmonella, E. Coli. Acinetobacter baumanii, Pseudomonas aeruginosa or Klebsiella pneumoniae.

Also provided are methods of overcoming acquired resistance to an antimicrobial agent that comprise conjugating the antimicrobial agent to a 2,3-pyrrolidinedione defined by the structure below:

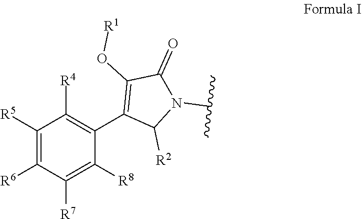

or a pharmaceutically acceptable salt or prodrug thereof, wherein
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl.

### BRIEF DESCRIPTION OF THE DRAWINGS

FIG. 1 illustrates the example vancomycin-2,3-pyrrolidinedione conjugates (Compounds 1-10) prepared in the Examples.

FIG. 2 is a plot showing the time-dependent killing of S. aureus (strain HG003) by vancomycin and an example vancomycin-2-3-pyrrolidinedione conjugate (Compound 4).

FIG. 3 is a plot showing the time-dependent killing of MRSA (strain LAC) by vancomycin and an example vancomycin-2-3-pyrrolidinedione conjugate (Compound 4).

FIG. 4A compares the activity of vancomycin and Compound 4 against vanA-resistant Enterococcus spp.

FIG. 4B compares the activity of vancomycin and Compound 4 against vanB-resistant Enterococcus spp.

FIG. 4C compares the activity of vancomycin and Compound 4 against vanC1-resistant Enterococcus spp.

FIG. 4D compares the activity of vancomycin and Compound 4 against vancomycin-susceptible Enterococcus spp.

FIG. 5 is a plot showing the effect of a single dose of different concentrations of Compound 4 on the concentration of bacteria in the supernatant over a methicillin-susceptible Staphylococcus aureus (MSSA ATCC 25923).

FIG. 6 is a plot showing the time-dependent biofilm population following treatment of a methicillin-susceptible Staphylococcus aureus (MSSA ATCC 25923) biofilm with Compound 4.

FIG. 7 is a plot showing the MIC (in μg/mL) of Compound 4, vancomycin, and varying ratios of Compound 4 and vancomycin.

FIG. 8 is a plot comparing the efficacy of linezolid, Compound 4 (AV-0273), and Compound 8 (AV-0267) in the S. aureus VRS-2 thigh infection model, CFU/g thigh. Test animals were rendered neutropenic with cyclophosphamide administration, 150 mg/kg on Day −4 then 100 mg/kg on Day −1 prior to infection on Day 0. On Day 0, animals were intramuscularly inoculated with 8.4×10⁴ CFU/mouse (0.1 mL/animal) of the S. aureus VRS-2 strain. The vehicle and the test articles, Compound 4 (AV-0273) and Compound 8 (AV-0267), both at 100 mg/kg, were each intraperitoneally (IP) administered twice with a 12 h interval (BID q12h) at 2 and 14 h after infection. The reference standard, linezolid at 50 mg/kg, was orally (PO) administered BID q12 h starting at 2 h post-infection. Animals were sacrificed at 2 or 26 h post-infection, and the thigh tissues were harvested and weighed from each of the test animals. The bacterial counts (CFU/g thigh) of thigh tissue homogenates were measured.

FIG. 9 is a plot comparing the efficacy of linezolid Compound 4 (AV-0273), and Compound 8 (AV-0267) in the S. aureus VRS-2 thigh infection model, CFU/g thigh, change in bacterial counts relative to baseline count at 26h post-infection. Data represent the change in bacterial counts in thigh tissue following test article treatment relative to the initial 2 h CFU/g thigh or CFU/thigh at the time of dosing (baseline counts). Test animals were rendered neutropenic with cyclophosphamide administration, 150 mg/kg on Day −4 then 100 mg/kg on Day −1 prior to infection on Day 0. On Day 0, animals were intramuscularly inoculated with 8.4×10⁴ CFU/mouse (0.1 mL/animal) of the S. aureus VRS-2 strain. The vehicle and the test articles, Compound 4 (AV-0273) and Compound 8 (AV-0267), both at 100 mg/kg, were each intraperitoneally (IP) administered twice with a 12 h interval (BID q12 h) at 2 and 14 h after infection. The reference standard, linezolid at 50 mg/kg, was orally (PO) administered BID q12 h starting at 2 h post-infection. Animals were sacrificed at 2 or 26 h post-infection, and the thigh tissues were harvested and weighed from each of the test animals. The bacterial counts (CFU/g thigh and CFU/thigh) of thigh tissue homogenates were compared.

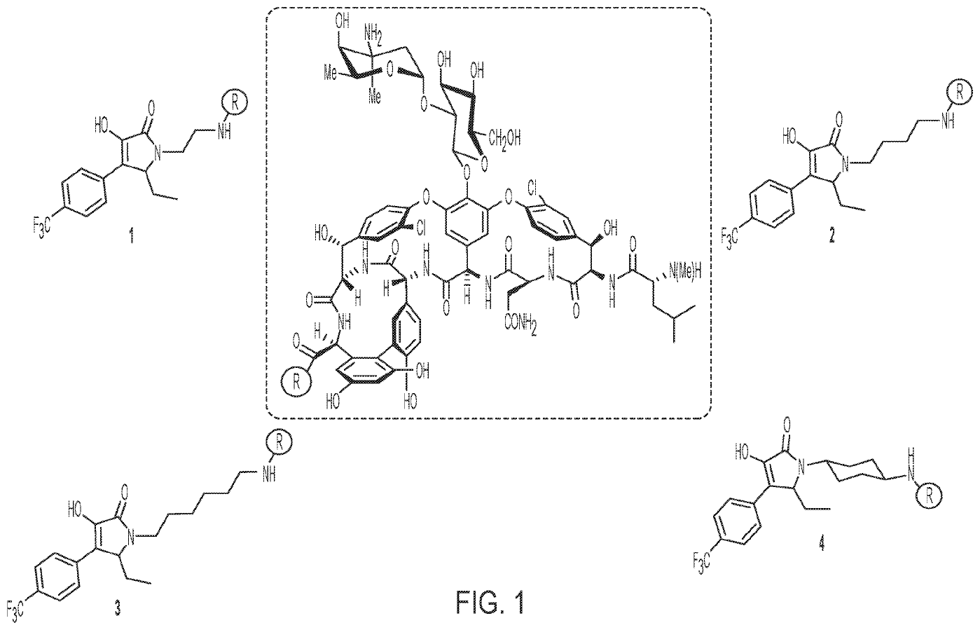

### DETAILED DESCRIPTION

### Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cₙ-Cₘ indicates in each case the possible number of carbon atoms in the group.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., biofilm growth). It is understood that this is typically in relation to some standard or expected value, in other words it is relative, but that it is not always necessary for the standard or relative value to be referred to. For example, “reducing the biofilm component of a chronic bacterial infection” can refer to reducing the rate of growth of a biofilm component of the chronic bacterial infection relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other forms of the word, such as “treated” or “treatment,” is meant to administer a composition or to perform a method in order to reduce, prevent, inhibit, or eliminate a particular characteristic or event (e.g., a biofilm). The term “control” is used synonymously with the terms “treat” and “modulate.”

“Biofilm” or “biofilms” refer to communities of microorganisms that are attached to a substrate. The microorganisms often excrete a protective and adhesive matrix of polymeric compounds. They often have structural heterogeneity, genetic diversity, and complex community interactions. “Biofilm preventing”, “biofilm removing”, “biofilm inhibiting”, “biofilm reducing”, “biofilm resistant”, “biofilm controlling” or “antifouling” refer to prevention of biofilm formation, inhibition of the establishment or growth of a biofilm, or decrease in the amount of organisms that attach and/or grow upon a substrate, up to and including the complete removal of the biofilm.

As used herein, a “substrate” can include any living or nonliving structure. For example, biofilms often grow on synthetic materials submerged in an aqueous solution or exposed to humid air, but they also can form as floating mats on a liquid surface, in which case the microorganisms are adhering to each other or to the adhesive matrix characteristic of a biofilm.

An “effective amount” of a biofilm preventing, removing or inhibiting composition is that amount which is necessary to carry out the composition's function of preventing, removing or inhibiting a biofilm.

The term “alkyl,” as used herein, refers to saturated straight, branched, cyclic, primary, secondary or tertiary hydrocarbons, including those having 1 to 20 atoms. In some embodiments, alkyl groups will include C₁-C₁₂, C₁-C₁₀, C₁-C₈, C₁-C₆, C₁-C₅, C₁-C₄, C₁-C₃, C₁-C₂, or C₁ alkyl groups. Examples of C₁-C₁₀ alkyl groups include, but are not limited to, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl groups, as well as their isomers. Examples of C₁-C₄-alkyl groups include, for example, methyl, ethyl, propyl, 1-methylethyl, butyl, 1-methylpropyl, 2-methylpropyl and 1,1-dimethylethyl groups.

Cyclic alkyl groups or “cycloalkyl” groups, which are encompassed alkyl, include cycloalkyl groups having from 3 to 10 carbon atoms. Cycloalkyl groups can include a single ring, or multiple condensed rings. In some embodiments, cycloalkyl groups include C₃-C₄, C₄-C₇, C₅-C₇, C₄-C₆, or C₅-C₆ cyclic alkyl groups. Non-limiting examples of cycloalkyl groups include adamantyl, cyclopropyl, cyclobutyl, cyclopentyl, cyclohexyl, cycloheptyl, cyclooctyl and the like.

Alkyl groups can be unsubstituted or substituted with one or more moieties selected from the group consisting of alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, phosphoric acid, phosphate, phosphonate, or any other viable functional group that does not inhibit the biological activity of the compounds of the invention, either unprotected, or protected as necessary, as known to those skilled in the art, for example, as described in Greene, et al., Protective Groups in Organic Synthesis, John Wiley and Sons, Third Edition, 1999, hereby incorporated by reference.

Terms including the term “alkyl,” such as “alkylcycloalkyl,” “cycloalkylalkyl,” “alkylamino,” or “dialkylamino,” will be understood to comprise an alkyl group as defined above linked to another functional group, where the group is linked to the compound through the last group listed, as understood by those of skill in the art.

The term “alkenyl,” as used herein, refers to both straight and branched carbon chains which have at least one carbon-carbon double bond. In some embodiments, alkenyl groups can include C₂-C₂₀ alkenyl groups. In other embodiments, alkenyl can include C₂-C₁₂, C₂-C₁₀, C₂-C₈, C₂-C₆ or C₂-C₄ alkenyl groups. In one embodiment of alkenyl, the number of double bonds is 1-3, in another embodiment of alkenyl, the number of double bonds is one or two. Other ranges of carbon-carbon double bonds and carbon numbers are also contemplated depending on the location of the alkenyl moiety on the molecule. “C₂-C₁₀-alkenyl” groups may include more than one double bond in the chain. The one or more unsaturations within the alkenyl group may be located at any position(s) within the carbon chain as valence permits. In some embodiments, when the alkenyl group is covalently bound to one or more additional moieties, the carbon atom(s) in the alkenyl group that are covalently bound to the one or more additional moieties are not part of a carbon-carbon double bond within the alkenyl group. Examples of alkenyl groups include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl groups.

The term “alkynyl,” as used herein, refers to both straight and branched carbon chains which have at least one carbon-carbon triple bond. In one embodiment of alkynyl, the number of triple bonds is 1-3; in another embodiment of alkynyl, the number of triple bonds is one or two. In some embodiments, alknyl groups include from C₂-C₂₀ alkynyl groups. In other embodiments, alkynyl groups may include C₂-C₁₂, C₂-C₁₀, C₂-C₈, C₂-C₆ or C₂-C₄ alkynyl groups. Other ranges of carbon-carbon triple bonds and carbon numbers are also contemplated depending on the location of the alkenyl moiety on the molecule. For example, the term “C₂-C₁₀-alkynyl” as used herein refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl, and 4-methylpent-2-yn-5-yl groups.

The term “haloalkyl,” as used herein refers to an alkyl group, as defined above, which is substituted by one or more halogen atoms. In some instances, the haloalkyl group can be an alkyl group substituted by one or more fluorine atoms. In certain instances, the haloalkyl group can be a perfluorinated alkyl group. For example, C₁-C₄-haloalkyl includes, but is not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, and pentafluoroethyl.

The term “haloalkenyl,” as used herein, refers to an alkenyl group, as defined above, which is substituted by one or more halogen atoms.

The term “haloalkynyl,” as used herein, refers to an alkynyl group, as defined above, which is substituted by one or more halogen atoms.

The term “alkoxy,” as used herein, refers to alkyl-O—, wherein alkyl refers to an alkyl group, as defined above. Similarly, the terms “alkenyloxy,” “alkynyloxy,” “haloalkoxy,” “haloalkenyloxy,” “haloalkynyloxy,” “cycloalkoxy,” “cycloalkenyloxy,” “halocycloalkoxy,” and “halocycloalkenyloxy” refer to the groups alkenyl-O—, alkynyl-O—, haloalkyl-O—, haloalkenyl-O—, haloalkynyl-O—, cycloalkyl-O—, cycloalkenyl-O—, halocycloalkyl-O—, and halocycloalkenyl-O—, respectively, wherein alkenyl, alkynyl, haloalkyl, haloalkenyl, haloalkynyl, cycloalkyl, cycloalkenyl, halocycloalkyl, and halocycloalkenyl are as defined above. Examples of C₁-C₆-alkoxy include, but are not limited to, methoxy, ethoxy, C₂H₅—CH₂O—, (CH₃)₂CHO—, n-butoxy, C₂H₅—CH(CH₃)O—, (CH₃)₂CH—CH₂O—. (CH₃)₃CO—, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy.

The term “alkylthio,” as used herein, refers to alkyl-S—, wherein alkyl refers to an alkyl group, as defined above. Similarly, the terms “haloalkylthio,” “cycloalkylthio,” and the like, refer to haloalkyl-S— and cycloalkyl-S— where haloalkyl and cycloalkyl are as defined above.

The term “alkylsulfinyl,” as used herein, refers to alkyl-S(O)—, wherein alkyl refers to an alkyl group, as defined above. Similarly, the term “haloalkylsulfinyl” refers to haloalkyl-S(O)— where haloalkyl is as defined above.

The term “alkylsulfonyl,” as used herein, refers to alkyl-S(O)₂—, wherein alkyl is as defined above. Similarly, the term “haloalkylsulfonyl” refers to haloalkyl-S(O)₂— where haloalkyl is as defined above.

The terms “alkylamino” and “dialkylamino,” as used herein, refer to alkyl-NH— and (alkyl)₂N— groups, where alkyl is as defined above. Similarly, the terms “haloalkylamino” and “halodialkylamino” refer to haloalkyl-NH— and (haloalkyl)₂-NH—, where haloalkyl is as defined above.

The terms “alkylcarbonyl,” “alkoxycarbonyl,” “alkylaminocarbonyl,” and “dialkylaminocarbonyl,” as used herein, refer to alkyl-C(O)—, alkoxy-C(O)—, alkylamino-C(O)— and dialkylamino-C(O)— respectively, where alkyl, alkoxy, alkylamino, and dialkylamino are as defined above. Similarly, the terms “haloalkylcarbonyl,” “haloalkoxycarbonyl,” “haloalkylaminocarbonyl,” and “dihaloalkylaminocarbonyl,” as used herein, refer to the groups haloalkyl-C(O)—, haloalkoxy-C(O)—, haloalkylamino-C(O)—, and dihaloalkylamino-C(O)—, where haloalkyl, haloalkoxy, haloalkylamino, and dihaloalkylamino are as defined above.

The term “aryl,” as used herein, refers to a monovalent aromatic carbocyclic group of from 6 to 14 carbon atoms. Aryl groups can include a single ring or multiple condensed rings. In some embodiments, aryl groups include C₆-C₁₀ aryl groups. Aryl groups include, but are not limited to, phenyl, biphenyl, naphthyl, tetrahydronaphtyl, phenylcyclopropyl and indanyl. Aryl groups may be unsubstituted or substituted by one or more moieties selected from halogen, cyano, nitro, hydroxy, mercapto, amino, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, cycloalkoxy, cycloalkenyloxy, halocycloalkoxy, halocycloalkenyloxy, alkylthio, haloalkylthio, cycloalkylthio, halocycloalkylthio, alkylsulfinyl, alkenylsulfinyl, alkynyl-sulfinyl, haloalkylsulfinyl, haloalkenylsulfinyl, haloalkynylsulfinyl, alkylsulfonyl, alkenylsulfonyl, alkynylsulfonyl, haloalkyl-sulfonyl, haloalkenylsulfonyl, haloalkynylsulfonyl, alkylamino, alkenylanino, alkynylamino, di(alkyl)amino, di(alkenyl)-amino, di(alkynyl)amino, or trialkylsilyl.

The term “alkylaryl,” as used herein, refers to an aryl group that is bonded to a parent compound through a diradical alkylene bridge, (—CH₂—)ₙ, where n is 1-12 and where “aryl” is as defined above.

The term “alkylcycloalkyl,” as used herein, refers to a cycloalkyl group that is bonded to a parent compound through a diradical alkylene bridge, (—CH₂—)ₙ, where n is 1-12 and where “cycloalkyl” is as defined above. The term “cycloalkylalkyl,” as used herein, refers to a cycloalkyl group, as defined above, which is substituted by an alkyl group, as defined above.

The term “heteroalkyl,” as used herein, refers to an alkyl group, as described above, which includes one or more heteroatoms (e.g., from one to four heteroatoms) within the carbon backbone. In some cases, the heteroatom(s) incorporated into the carbon backbone are oxygen, nitrogen, sulfur, or combinations thereof. The terms “heteroalkenyl” and “heteroalkynyl,” as used herein, likewise refer to alkenyl and alkynyl groups respectively which include one or more heteroatoms (e.g., from one to four heteroatoms, such as oxygen, nitrogen, sulfur, or combinations thereof) within their carbon backbone.

The term “heteroaryl,” as used herein, refers to a monovalent aromatic group of from 1 to 15 carbon atoms (e.g., from 1 to 10 carbon atoms, from 2 to 8 carbon atoms, from 3 to 6 carbon atoms, or from 4 to 6 carbon atoms) having one or more heteroatoms within the ring. The heteroaryl group can include from 1 to 4 heteroatoms, from 1 to 3 heteroatoms, or from 1 to 2 heteroatoms. In some cases, the heteroatom(s) incorporated into the ring are oxygen, nitrogen, sulfur, or combinations thereof. When present, the nitrogen and sulfur heteroatoms may optionally be oxidized. Heteroaryl groups can have a single ring (e.g., pyridyl or furyl) or multiple condensed rings provided that the point of attachment is through a heteroaryl ring atom. Preferred heteroaryls include pyridyl, piridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, and benzothiophenyl. Heteroaryl rings may be unsubstituted or substituted by one or more moieties as described for aryl above.

The term “alkylheteroaryl,” as used herein, refers to a heteroaryl group that is bonded to a parent compound through a diradical alkylene bridge, (—CH₂—)ₙ, where n is 1-12 and where “heteroaryl” is as defined above.

The terms “cycloheteroalkyl,” “heterocyclyl,” “heterocyclic,” and “heterocyclo” are used herein interchangeably, and refer to fully saturated or unsaturated, cyclic groups, for example, 3 to 7 membered monocyclic or 4 to 7 membered monocyclic; 7 to 11 membered bicyclic, or 10 to 15 membered tricyclic ring systems, having one or more heteroatoms within the ring. The heterocyclyl group can include from 1 to 4 heteroatoms, from 1 to 3 heteroatoms, or from 1 to 2 heteroatoms. In some cases, the heteroatom(s) incorporated into the ring are oxygen, nitrogen, sulfur, or combinations thereof. When present, the nitrogen and sulfur heteroatoms may optionally be oxidized, and the nitrogen heteroatoms may optionally be quaternized. The heterocyclyl group may be attached at any heteroatom or carbon atom of the ring or ring system and may be unsubstituted or substituted by one or more moieties as described for aryl groups above.

Exemplary monocyclic heterocyclic groups include, but are not limited to, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, triazinyl, and the like.

Exemplary bicyclic heterocyclic groups include, but are not limited to, indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl]or furo[2,3-b]pyridinyl), dihydroisoindolyl, dihydroquinazolinyl (such as 3,4-dihydro-4-oxo-quinazolinyl), tetrahydroquinolinyl and the like.

Exemplary tricyclic heterocyclic groups include carbazolyl, benzidolyl, phenanthrolinyl, acridinyl, phenanthridinyl, xanthenyl, and the like.

The term “alkylheterocyclyl” and “alkylcycloheteroalkyl” are used herein interchangeably, and refer to a heterocyclyl group that is bonded to a parent compound through a diradical alkylene bridge, (—CH₂—)ₙ, where n is 1-12 and where “heterocyclyl” is as defined above. The term “heterocyclylalkyl,” as used herein, refers to a heterocyclyl group, as defined above, which is substituted by an alkyl group, as defined above.

The term “halogen,” as used herein, refers to the atoms fluorine, chlorine, bromine and iodine. The prefix halo- (e.g., as illustrated by the term haloalkyl) refers to all degrees of halogen substitution, from a single substitution to a perhalo substitution (e.g., as illustrated with methyl as chloromethyl (—CH₂Cl), dichloromethyl (—CHCl₂), trichloromethyl (—CCl₃)).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

### Stereoisomers and Polymorphic Forms

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The compounds described herein can exist and be isolated as optically active and racemic forms. The compounds can have one or more chiral centers, including at a sulfur atom, and thus exist as one or more stereoisomers. Where compounds include n chiral centers, the compounds can comprise up to 2ⁿ optical isomers. Such stereoisomer-containing compounds can exist as a single enantiomer, a mixture of enantiomers, a mixture of diastereomers, or a racemic mixture.

The optically active forms can be prepared by, for example, resolution of the racemic forms by selective crystallization techniques, by synthesis from optically active precursors, by chiral synthesis, by chromatographic separation using a chiral stationary phase or by enzymatic resolution.

The compounds can also be present in different solid forms, including different crystalline forms (i.e., different crystalline polymorphs of the compounds) or as an amorphous solid. In addition, the compounds can exist as hydrates or solvates, in which a certain stoichiometric amount of water or a solvent is associated with the molecule in the crystalline form. In some embodiments, the compositions described herein can include up to 15% (w/w), up to 20% (w/w), or up to 30% (w/w) of a particular solid form of the compounds described herein, based on the total weight of the composition.

### Pharmaceutically Acceptable Salts

The compounds described herein can also be provided as pharmaceutically acceptable salts (e.g., acid or base salts) where applicable, of the compounds described herein. Pharmaceutically acceptable salts are known in the art. See, for example. Remington's Pharmaceutical Sciences, 20th ed., Lippincott Williams & Wilkins, Baltimore, MD, 2000, p. 704.

The term “acid salt” contemplates salts of the compounds with all pharmaceutically acceptable inorganic or organic acids. Inorganic acids include mineral acids such as hydrohalic acids such as hydrobromic acid and hydrochloric acid, sulfuric acid, phosphoric acids and nitric acid. Organic acids include all pharmaceutically acceptable aliphatic, alicyclic and aromatic carboxylic acids, dicarboxylic acids, tricarboxylic acids and fatty acids. In one embodiment of the acids, the acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, acetic acid, propionic acid, isopropionic acid, valeric acid, α-hydroxy acids such as glycolic acid and lactic acid, chloroacetic acid, benzoic acid, methane sulfonic acid, and salicylic acid. Examples of dicarboxylic acids include oxalic acid, malic acid, succinic acid, tartaric acid, fumaric acid, and maleic acid. An example of a tricarboxylic acid is citric acid. Fatty acids include all pharmaceutically acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

The term “base salt” contemplates salts of the compounds with all pharmaceutically acceptable inorganic or organic bases, including hydroxides, carbonates or bicarbonates of alkali metal or alkaline earth metals. Salts formed with such bases include, for example, the alkali is metal and alkaline earth metal salts, including, but not limited to, as the lithium, sodium, potassium, magnesium or calcium salts. Salts formed with organic bases include the common hydrocarbon and heterocyclic amine salts, which include, for example, ammonium salts (NH₄⁺), alkylammonium salts, and dialkylammonium salts, as well as salts of cyclic amines such as the morpholine and piperidine salts.

### Prodrugs

The compounds described herein can also be provided as pharmaceutically acceptable prodrugs. Prodrugs of are compounds that, when metabolized in vivo, undergo conversion to compounds described herein having the desired pharmacological activity. Prodrugs can be prepared by replacing appropriate functionalities present in the compounds described herein with “pro-moieties” as described, for example, in H. Bundgaar, Design of Prodrugs (1985). Examples of prodrugs include ester, ether or amide derivatives of the compounds described herein, as well as their pharmaceutically acceptable salts. For further discussions of prodrugs, see, for example, T. Higuchi and V. Stella “Pro-drugs as Novel Delivery Systems,” ACS Symposium Series 14 (1975) and E. B. Roche ed., Bioreversible Carriers in Drug Design (1987).

### Compounds

Provided herein are compounds that can exhibit activity as biofilm modulating agents (e.g., activity as biofilm inhibitors and/or activity as biofilm dispersal agents). The compounds can exhibit potent activity against Gram positive biofilms. The compounds can also exhibit activity against Gram negative biofilms. In some cases, the compounds can exhibit both biofilm modulation properties and antimicrobial activity.

For example, provided herein are compounds defined by Formula I

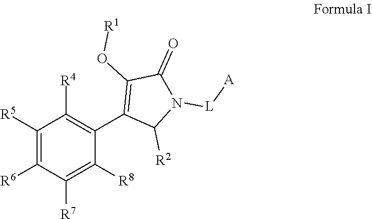

or a pharmaceutically acceptable salt or prodrug thereof, wherein
L is absent, or represents a bivalent linking group;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl.

In some embodiments of Formula I, R¹ is hydrogen or a C₁-C₄ alkyl group optionally substituted with one or more substituents individually chosen from R⁹. In certain embodiments, R¹ is hydrogen. In other embodiments, R¹ is an unsubstituted C₁-C₄ alkyl group.

In some embodiments of Formula I, R² is a C₁-C₄ alkyl group optionally substituted with one or more substituents individually chosen from R⁹. In certain embodiments. R² is an unsubstituted C₁-C₄ alkyl group. In some examples, R² is methyl or ethyl.

In other embodiments of Formula I, R² can be an alkylaryl group optionally substituted with one or more substituents individually chosen from R⁹. In certain embodiments, R² can be a benzyl group optionally substituted with one or more substituents individually chosen from R⁹. In some examples, R² can be benzyl or hydroxybenzyl.

In some embodiments of Formula I, at least one of R⁴, R⁵, R⁶, R⁷, and R⁸ is not hydrogen. In some cases, R⁴, R⁵, R⁷, and R⁸ are hydrogen. In certain cases, R⁴, R⁵, R⁷, and R⁸ are hydrogen, and R⁶ is not hydrogen (e.g., the phenyl ring attached to the 2-position of the pyrrolidinone ring is para-substituted).

In some embodiments of Formula I, R⁶ can be an electron withdrawing group. For example, R⁶ can be chosen from halogen, —CN, —NO₂, haloalkyl, alkylcarbonyl, haloalkylcarbonyl, alkoxycarbonyl, and haloalkoxycarbonyl. In some embodiments, R⁶ can be a haloalkyl group. In certain embodiments, R¹ can be a perfluoroalkyl group (e.g., a —CF₃ group).

When present, the linking group can be any suitable group or moiety which is at minimum bivalent, and connects the two radical moieties to which the linking group is attached in the compounds described herein. The linking group can be composed of any assembly of atoms, including oligomeric and polymeric chains. In some cases, the total number of atoms in the linking group can be from 3 to 50 atoms (e.g., from 3 to 40 atoms, from 3 to 30 atoms, from 3 to 25 atoms, from 3 to 20 atoms, from 3 to 15 atoms, from 3 to 10 atoms, or from 3 and 5 atoms).

In some embodiments, the linking group can be, for example, an alkyl, alkoxy, alkylaryl, alkylheteroaryl, alkylcycloalkyl, alkylheterocycloalkyl, alkylthio, alkylsulfinyl, alkylsulfonyl, alkylamino, dialkylamino, alkylcarbonyl, alkoxycarbonyl, alkylaminocarbonyl, dialkylaminocarbonyl, or polyamino group. In some embodiments, the linking group can comprises one of the groups above joined to one or both of the moieties to which it is attached by a functional group. Examples of suitable functional groups include, for example, secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, or —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. For example, in some embodiments, the linking group can comprise an alkyl group (e.g., a C₁-C₁₂ alkyl group, a C₁-C₈ alkyl group, or a C₁-C₆ alkyl group) bound to one or both of the moieties to which it is attached via an ester (—COO—, —CH₂O₂C—, CHRO₂C—), a secondary amide (—CONH—), or a tertiary amide (—CONR—), wherein R is an alkyl group, an aryl group, or a heterocyclic group. In certain embodiments, the linking group can be chosen from one of the following:

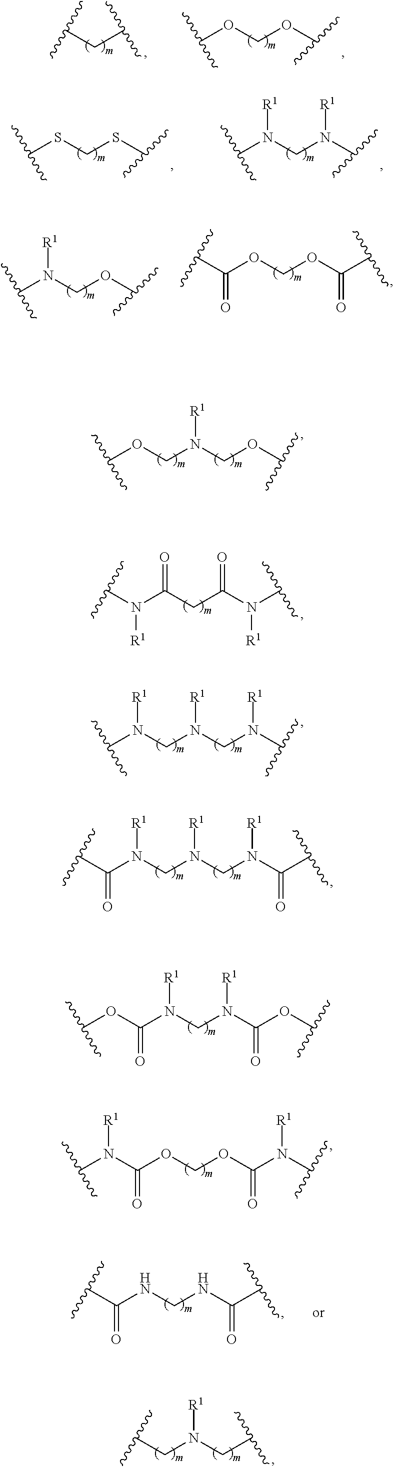

where m is an integer from 1 to 12 and R¹ is, independently for each occurrence, hydrogen, an alkyl group, an aryl group, or a heterocyclic group.

If desired, the linker can serve to modify the solubility of the compounds described herein. In some embodiments, the linker is hydrophilic. In some embodiments, the linker can be an alkyl group, an alkylaryl group, an oligo- or polyalkylene oxide chain (e.g., an oligo- or polyethylene glycol chain), or an oligo- or poly(amino acid) chain.

In certain embodiments, the linker can be cleavable (e.g., cleavable by hydrolysis under physiological conditions, enzymatically cleavable, or a combination thereof). Examples of cleavable linkers include a hydrolysable linker, a pH cleavage linker, an enzyme cleavable linker, or disulfide bonds that are cleaved through reduction by free thiols and other reducing agents; peptide bonds that are cleaved through the action of proteases and peptidase; nucleic acid bonds cleaved through the action of nucleases; esters that are cleaved through hydrolysis either by enzymes or through the action of water in vivo; hydrazones, acetals, ketals, oximes, imine, aminals and similar groups that are cleaved through hydrolysis in the body; photo-cleavable bonds that are cleaved by the exposure to a specific wavelength of light; mechano-sensitive groups that are cleaved through the application of ultrasound or a mechanical strain (e.g., a mechanical strain created by a magnetic field on a magneto-responsive gel).

In other embodiments, the linker can be non-cleavable.

In some embodiments, L can comprise a positively charged moiety.

In some embodiments, the compound can be defined by Formula IA

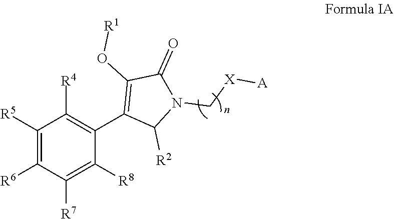

or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
n is an integer from 2 to 12;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

In some embodiments of Formula IA, R¹ is hydrogen or a C₁-C₄ alkyl group optionally substituted with one or more substituents individually chosen from R⁹. In certain embodiments, R¹ is hydrogen. In other embodiments, R¹ is an unsubstituted C₁-C₄ alkyl group.

In some embodiments of Formula IA, R² is a C₁-C₄ alkyl group optionally substituted with one or more substituents individually chosen from R⁹. In certain embodiments, R² is an unsubstituted C₁-C₄ alkyl group. In some examples, R² is methyl or ethyl.

In other embodiments of Formula IA, R² can be an alkylaryl group optionally substituted with one or more substituents individually chosen from R⁹. In certain embodiments, R² can be a benzyl group optionally substituted with one or more substituents individually chosen from R⁹. In some examples, R² can be benzyl or hydroxybenzyl.

In some embodiments of Formula IA, at least one of R⁴, R⁵, R⁶, R⁷, and R⁸ is not hydrogen. In some cases, R⁴, R⁵, R⁶, R⁷, and R⁸ are hydrogen. In certain cases, R⁴, R⁵, R⁷, and R⁸ are hydrogen, and R⁶ is not hydrogen (e.g., the phenyl ring attached to the 2-position of the pyrrolidinone ring is para-substituted).

In some embodiments of Formula IA, R⁶ can be an electron withdrawing group. For example, R⁶ can be chosen from halogen, —CN, —NO₂, haloalkyl, alkylcarbonyl, haloalkylcarbonyl, alkoxycarbonyl, and haloalkoxycarbonyl. In some embodiments, R⁶ can be a haloalkyl group. In certain embodiments, R⁶ can be a perfluoroalkyl group (e.g., a —CF₃ group).

In some embodiments, the compound can be defined by Formula IB

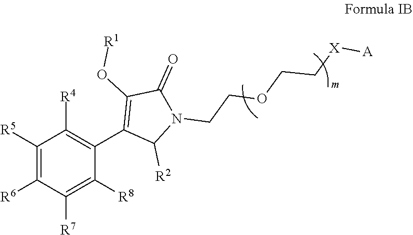

or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
m is an integer from 1 to 20;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

In some embodiments of Formula IB, R¹ is hydrogen or a C₁-C₄ alkyl group optionally substituted with one or more substituents individually chosen from R⁹. In certain embodiments, R¹ is hydrogen. In other embodiments, R¹ is an unsubstituted C₁-C₄ alkyl group.

In some embodiments of Formula IB, R² is a C₁-C₄ alkyl group optionally substituted with one or more substituents individually chosen from R⁹. In certain embodiments, R² is an unsubstituted C₁-C₄ alkyl group. In some examples, R² is methyl or ethyl.

In other embodiments of Formula IB, R² can be an alkylaryl group optionally substituted with one or more substituents individually chosen from R⁹. In certain embodiments, R² can be a benzyl group optionally substituted with one or more substituents individually chosen from R⁹. In some examples, R² can be benzyl or hydroxybenzyl.

In some embodiments of Formula IB, at least one of R⁴, R⁵, R⁶, R⁷, and R⁸ is not hydrogen. In some cases, R⁴, R⁵, R⁷, and R⁸ are hydrogen. In certain cases, R⁴, R⁵, R⁷, and R⁸are hydrogen, and R⁶ is not hydrogen (e.g., the phenyl ring attached to the 2-position of the pyrrolidinone ring is para-substituted).

In some embodiments of Formula IB, R⁶ can be an electron withdrawing group. For example, R⁶ can be chosen from halogen, —CN, —NO₂, haloalkyl, alkylcarbonyl, haloalkylcarbonyl, alkoxycarbonyl, and haloalkoxycarbonyl. In some embodiments, R⁶ can be a haloalkyl group. In certain embodiments, R⁶ can be a perfluoroalkyl group (e.g., a —CF₃ group).

In some embodiments, the compound can be defined by Formula IC or Formula ID

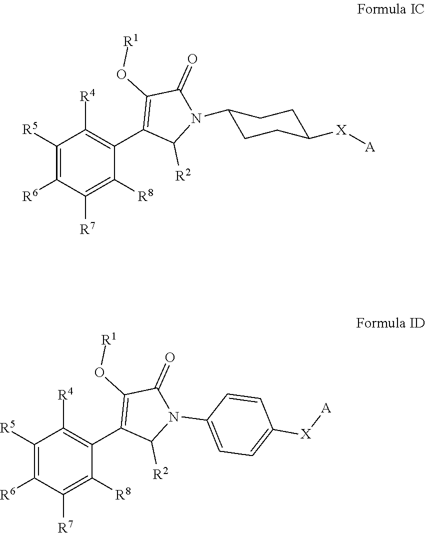

or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
s A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

In some embodiments of Formula IC and ID, R¹ is hydrogen or a C₁-C₄ alkyl group optionally substituted with one or more substituents individually chosen from R⁹.

In some embodiments of Formula IC and ID, R² is a C₁-C₄ alkyl group optionally substituted with one or more substituents individually chosen from R⁹. In certain embodiments, R² is an unsubstituted C₁-C₄ alkyl group. In some examples, R² is methyl or ethyl.

In other embodiments of Formula IC and ID, R² can be an alkylaryl group optionally substituted with one or more substituents individually chosen from R⁹. In certain embodiments, R² can be a benzyl group optionally substituted with one or more substituents individually chosen from R⁹. In some examples, R² can be benzyl or hydroxybenzyl.

In some embodiments of Formula IC and ID, at least one of R⁴, R⁵, R⁶, R⁷, and R⁸ is not hydrogen. In some cases, R⁴, R⁵, R⁶, R⁷, and R⁸ are all hydrogen.

In some embodiments of Formula IC and ID, R⁶ is an electron withdrawing group. In certain cases, R⁶ can be a haloalkyl. In certain cases, R⁶ can be a perfluoroalkyl group (e.g., —CF₃).

In some embodiments of Formula I, IA, IB, IC, and ID, A can comprise an antibacterial agent. In some cases, the antibacterial agent can be an anti-bacterial agent that acts via an extracellular mechanism of action. For example, the anti-bacterial agent can target bacterial cell walls. Examples of such antibacterial agents include f-lactam antibiotics, glycopeptide antibiotics, and polypeptide antibiotics.

In some embodiments of Formula I, IA, IB, IC, and ID, A can comprise a β-lactam antibiotic.

In some embodiments of Formula I, IA, IB, IC, and ID, A can comprise a penicillin. Penicillins include, but are not limited to, amoxicillin, ampicillin, azlocillin, bacampicillin, carbenicillin, cloxacillin, dicloxacillin, flucloxacillin, mezlocillin, meticillin, nafcillin, oxacillin, penicillin, piperacillin and ticarcillin.

In some embodiments of Formula I, IA, IB, IC, and ID, A can comprise a cephalosporins. Cephalosporins include, but are not limited to, cefadroxil, cefazolin, cefalotin (cefalothin), cefalexin, cefaclor, cefamandole, cefoxitin, cefprozil, loracarbef, cefuroxime, cefixime, cefdinir, cefditoren, cefoperazone, cefotaxime, cefpodoxime, ceftazidime, ceftibuten, ceftizoxime, cefiriaxone, cefepime, cefpirome, and ceftobiprole.

In some embodiments of Formula I, IA, IB, IC, and ID, A can comprise a monobactams. Monobactams include, but are not limited to, aztreonam, tigemonam, nocardicin A, and tabtoxin.

In some embodiments of Formula I, IA, IB, IC, and ID, A can comprise a carbapenem. Carbapenems include, but are not limited to, ertapenem, doripenem, imipenem/cilstatin, and meropenem.

In some embodiments of Formula I, IA, IB, IC, and ID, A can comprise a glycopeptide antibiotic. For example, in some cases, A can comprise teicoplanin, vancomycin, telavancin, dalbavancin, or oritavancin.

In some embodiments of Formula I, IA, IB, IC, and ID, A can comprise a polypeptide antibiotic. For example, in some cases, A can comprise bacitracin, colistin, or polymyxin B.

In certain embodiments of Formula I, IA, IB, and IC, A can comprise a glycopeptide antibiotic, such as vancomycin.

### Compositions

Also provided are compositions that include one or more of the compounds described herein. In some embodiments, biofilm preventing, removing or inhibiting compositions are provided, comprising a carrier and an effective amount of a compound described herein.

In some embodiments, the carrier can be a pharmaceutically acceptable carrier. A “pharmaceutically acceptable carrier” as used herein refers to a carrier that, when combined with a compound described herein, facilitates the application or administration of that compound described herein for its intended purpose (e.g., to prevent or inhibit biofilm formation, or remove an existing biofilm). The compound described herein may be formulated for administration in a pharmaceutically acceptable carrier in accordance with known techniques. See, e.g., Remington, The Science And Practice of Pharmacy (9th Ed. 1995). The pharmaceutically acceptable carrier can, of course, also be acceptable in the sense of being compatible with any other ingredients in the composition.

The carrier may be a solid or a liquid, or both, and is preferably formulated with the a compound described herein as a unit-dose composition, for example, a tablet, which may contain from 0.01 or 0.5% to 95% or 99% by weight of the a compound described herein. One or more a compounds described herein can be included in the compositions, which may be prepared by any of the well-known techniques of pharmacy comprising admixing the components, optionally including one or more accessory ingredients.

In general, compositions may be prepared by uniformly and intimately admixing the a compound described herein with a liquid or finely divided solid carrier, or both, and then, if necessary, shaping the resulting mixture. For example, a tablet may be prepared by compressing or molding a powder or granules containing the a compound described herein, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Compositions can be formulated to be suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) or transdermal administration, although the most suitable route in any given case will depend on the nature and severity of the condition being treated and on the nature of the particular compound that is being used.

Compositions suitable for oral administration may be presented in discrete units, such as capsules, cachets, lozenges, or tablets, each containing a predetermined amount of the compound; as a powder or granules; as a solution or a suspension in an aqueous or non-aqueous liquid; or as an oil-in-water or water-in-oil emulsion. Such compositions may be prepared by any suitable method of pharmacy, which includes the step of bringing into association the compound and a suitable carrier (which may contain one or more accessory ingredients as noted above).

Compositions suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Compositions suitable for parenteral administration comprise sterile aqueous and non-aqueous injection solutions of the compound, which preparations are preferably isotonic with the blood of the intended recipient. These preparations may contain anti-oxidants, buffers, bacteriostats and solutes that render the composition isotonic with the blood of the intended recipient. Aqueous and non-aqueous sterile suspensions may include suspending agents and thickening agents. The compositions may be presented in unit/dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

For example, the composition can be an injectable, stable, sterile composition comprising a compound described herein in a unit dosage form in a sealed container. The compostions can be provided in the form of a lyophilizate that can be reconstituted with a suitable pharmaceutically acceptable carrier to form a liquid composition suitable for injection thereof into a subject. The unit dosage form can comprise from about 10 mg to about 10 grams of the compound. When the compound or salt is substantially water-insoluble, a sufficient amount of emulsifying agent that is physiologically acceptable may be employed in sufficient quantity to emulsify the compound or salt in an aqueous carrier. One such useful emulsifying agent is phosphatidyl choline.

Compositions suitable for rectal administration can be presented as unit dose suppositories. These may be prepared by mixing the active compound with one or more conventional solid carriers, for example, cocoa butter, and then shaping the resulting mixture.

Compositions suitable for topical application to the skin can take the form of an ointment, cream, lotion, paste, gel, spray, aerosol, or oil. Carriers that may be used include petroleum jelly, lanoline, polyethylene glycols, alcohols, transdermal enhancers, and combinations of two or more thereof.

Compositions suitable for transdermal administration can be presented as discrete patches adapted to remain in intimate contact with the epidermis of the recipient for a prolonged period of time. Compositions suitable for transdermal administration may also be delivered by iontophoresis and typically take the form of an optionally buffered aqueous solution of the active compound.

In some embodiments, the compositions described herein can further include one or more additional active agents, such as a biocide. A “biocide” as used herein refers to a substance with the ability to kill or to inhibit the growth of microorganisms (e.g., bacteria, fungal cells, protozoa, etc.), which is not compound described in the compounds section above. Common biocides include oxidizing and non-oxidizing chemicals.

In some embodiments, the compositions described herein can further include one or more antibiotics. An “antibiotic” as used herein is a type of “biocide.” Common antibiotics include aminoglycosides, carbacephems (e.g., loracarbef), carbapenems, cephalosporins, glycopeptides (e.g., teicoplanin and vancomycin), macrolides, monobactams (e.g., aztreonam) penicillins, polypeptides (e.g., bacitracin, colistin, polymyxin B), quinolones, sulfonamides, tetracyclines, etc. Antibiotics treat infections by either killing or preventing the growth of microorganisms. Many act to inhibit cell wall synthesis or other vital protein synthesis of the microorganisms.

Aminoglycosides are commonly used to treat infections caused by Gram-negative bacteria such as Escherichia coli and Klebsiella, particularly Pseudomonas aeroginosa. Examples of aminoglycosides include, but are not limited to amikacin, gentamicin, kanamycin, neomycin, netilmicin, streptomycin, tobramycin, and paromomycin.

Carbapenems are broad-spectrum antibiotics, and include, but are not limited to, ertapenem, doripenem, imipenem/cilstatin, and meropenem.

Cephalosporins include, but are not limited to, cefadroxil, cefazolin, cefalotin (cefalothin), cefalexin, cefaclor, cefamandole, cefoxitin, cefprozil, loracarbef, cefuroxime, cefixime, cefdinir, cefditoren, cefoperazone, cefotaxime, cefpodoxime, ceftazidime, ceftibuten, ceftizoxime, ceftriaxone, cefepime, cefpirome, and ceftobiprole.

Macrolides include, but are not limited to, azithromycin, clarithromycin, dirithromycin, erythromycin, roxithromycin, troleandomycin, telithromycin and spectinomycin.

Penicillins include, but are not limited to, amoxicillin, ampicillin, azlocillin, bacampicillin, carbenicillin, cloxacillin, dicloxacillin, flucloxacillin, mezlocillin, meticillin, nafcillin, oxacillin, penicillin, piperacillin and ticarcillin.

Quinolones include, but are not limited to, ciprofloxacin, enoxacin, gatifloxacin, gemifloxacin, levofloxacin, lomefloxacin, moxifloxacin, norfloxacin, ofloxacin and trovafloxacin.

Sulfonamides include, but are not limited to, mafenide, prontosil, sulfacetamide, sulfamethizole, sulfanilamide, sulfasalazine, sulfisoxazole, trimethoprim, and co-trimoxazole (trimethoprim-sulfamethoxazole).

Tetracyclines include, but are not limited to, demeclocycline, doxycycline, minocycline, oxytetracycline and tetracycline.

Other antibiotics include arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin (rifampicin), tinidazole, etc.

In some embodiments, the composition can be a dentifrice composition comprising one or more of the compounds described herein. A “dentifrice” is a substance that is used to clean the teeth. It may be in the form of, e.g., a paste or powder. Commonly known dentifrices include toothpaste, mouthwash, chewing gum, dental floss, and dental cream. Other examples of dentifrices include toothpowder, mouth detergent, troches, dental or gingival massage cream, dental strips, dental gels, and gargle tablets. Examples of dentifrice compositions comprising toothpaste and mouthwash are found in U.S. Pat. No. 6,861,048 (Yu et al.); U.S. Pat. No. 6,231,836 (Takhtalian et al.); and U.S. Pat. No. 6,331,291 (Glace et al.); each of which are incorporated by reference herein in their entirety.

Coating compositions are also provided. A “coating” as used herein is generally known. Any of a variety of organic and aqueous coating compositions, with or without pigments, may be modified to contain one or more compounds described herein. Examples of suitable coating compositions include, for example, the coating compositions described in U.S. Pat. Nos. 7,109,262, 6,964,989, 6,835,459, 6,677,035, 6,528,580, and 6,235,812, each incorporated by reference herein in their entirety.

In some examples, coating compositions can comprise (in addition to one or more compounds described herein) a film-forming resin, an aqueous or organic solvent that disperses the resin; and, optionally, at least one pigment. Other ingredients such as colorants, secondary pigments, stabilizers and the like can be included if desired. The one or more biofilm modulating compounds described herein may be dissolved or dispersed in the solvent and/or resin, so that the compound(s) are dispersed or distributed on the substrate an article coated by the coating composition. The resin may comprise, for example, a polymeric material. A polymeric material is a material that is comprised of large molecules made from associated smaller repeating structural units, often covalently linked. Common examples of polymeric materials are unsaturated polyester resins, and epoxy resins.

Any suitable article can be coated, in whole or in part, with the coating compositions described herein. Suitable articles include, but are not limited to, automobiles and airplanes (including substrates such as wing and propeller surfaces for aerodynamic testing), vessel hulls (including interior and exterior surfaces thereof), pressure vessels (including interior and exterior surfaces thereof), medical devices (e.g., implants), windmills, etc. Coating of the article with the composition can be carried out by any suitable means, such as by brushing, spraying, electrostatic deposition, dip coating, doctor blading, etc.

### Devices

Also provided are medical devices that comprise a medical device substrate and an effective amount of a compound described herein either coating the substrate, or incorporated into the substrate. The effective amount of the compound can be an effective amount to prevent or inhibit growth of a biofilm on the medical device substrate.

“Medical device” as used herein refers to an object that is inserted or implanted in a subject or applied to a surface of a subject. Common examples of medical devices include stents, fasteners, ports, catheters, scaffolds and grafts. A “medical device substrate” can be made of a variety of biocompatible materials, including, but not limited to, metals, ceramics, polymers, gels, and fluids not normally found within the human body. Examples of polymers useful in fabricating medical devices include such polymers as silicones, rubbers, latex, plastics, polyanhydrides, polyesters, polyorthoesters, polyamides, polyacrylonitrile, polyurethanes, polyethylene, polytetrafluoroethylene, polyethylenetetraphthalate, etc. Medical devices can also be fabricated using naturally-occurring materials or treated with naturally-occurring materials. Medical devices can include any combination of artificial materials, e.g., combinations selected because of the particular characteristics of the components. Medical devices can be intended for short-term or long-term residence where they are positioned. A hip implant is intended for several decades of use, for example. By contrast, a tissue expander may only be needed for a few months, and is removed thereafter.

Some examples of medical devices are found in U.S. Pat. No. 7,081,133 (Chinn et al.); U.S. Pat. No. 6,562,295 (Neuberger); and U.S. Pat. No. 6,387,363 (Gruskin); each incorporated by reference herein in their entirety.

### Methods of Use

Methods of controlling biofilm formation on a substrate are disclosed, comprising the step of administering a compound described herein to a substrate in an amount effective to inhibit biofilm formation.

A “substrate” as used herein is a base on which an organism, such as those commonly found in biofilms, may live. The term “substrate,” as used herein, refers to any substrate, whether in an industrial or a medical setting, that provides or can provide an interface between an object and a fluid, permitting at least intermittent contact between the object and the fluid. A substrate, as understood herein, further provides a plane whose mechanical structure, without further treatment, is compatible with the adherence of microorganisms. Substrates compatible with biofilm formation may be natural or synthetic, and may be smooth or irregular. Fluids contacting the substrates can be stagnant or flowing, and can flow intermittently or continuously, with laminar or turbulent or mixed flows. A substrate upon which a biofilm forms can be dry at times with sporadic fluid contact, or can have any degree of fluid exposure including total immersion. Fluid contact with the substrate can take place via aerosols or other means for air-borne fluid transmission.

Biofilm formation with health implications can involve those substrates in all health-related environments, including substrates found in medical environments and those substrates in industrial or residential environments that are involved in those functions essential to human well being, for example, nutrition, sanitation and the prevention of disease. Substrates found in medical environments include the inner and outer aspects of various instruments and devices, whether disposable or intended for repeated uses. Examples include the entire spectrum of articles adapted for medical use, including scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherably insertable central venous catheters, dialysis catheters, long term tunneled central venous catheters, peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, and the like. Other examples will be readily apparent to practitioners in these arts. Substrates found in the medical environment also include the inner and outer aspects of pieces of medical equipment, medical gear worn or carried by personnel in the health care setting. Such substrates can include counter tops and fixtures in areas used for medical procedures or for preparing medical apparatus, tubes and canisters used in respiratory treatments, including the administration of oxygen, of solubilized drugs in nebulizers and of anesthetic agents. Also included are those substrates intended as biological barriers to infectious organisms in medical settings, such as gloves, aprons and faceshields. Commonly used materials for biological barriers may be latex-based or non-latex based. Vinyl is commonly used as a material for non-latex surgical gloves. Other such substrates can include handles and cables for medical or dental equipment not intended to be sterile. Additionally, such substrates can include those non-sterile external substrates of tubes and other apparatus found in areas where blood or body fluids or other hazardous biomaterials are commonly encountered.

Substrates in contact with liquids are particularly prone to biofilm formation. As an example, those reservoirs and tubes used for delivering humidified oxygen to patients can bear biofilms inhabited by infectious agents. Dental unit waterlines similarly can bear biofilms on their substrates, providing a reservoir for continuing contamination of the system of flowing an aerosolized water used in dentistry. Sprays, aerosols and nebulizers are highly effective in disseminating biofilmfragments to a potential host or to another environmental site. It is especially important to health to prevent biofilmformation on those substrates from where biofilm fragments can be carried away by sprays, aerosols or nebulizers contacting the substrate.

Other substrates related to health include the inner and outer aspects of those articles involved in water purification, water storage and water delivery, and articles involved in food processing. Substrates related to health can also include the inner and outer aspects of those household articles involved in providing for nutrition, sanitation or disease prevention. Examples can include food processing equipment for home use, materials for infant care, tampons and toilet bowls. “Substrate” as used herein also refers to a living substrate, such as the inner ear of a patent.

Substrates can be smooth or porous, soft or hard. Substrates can include a drainpipe, glaze ceramic, porcelain, glass, metal, wood, chrome, plastic, vinyl, Formica® brand laminate, or any other material that may regularly come in contact with an aqueous solution in which biofilms may form and grow. The substrate can be a substrate commonly found on household items such as shower curtains or liners, upholstery, laundry, and carpeting.

A substrate on which biofilm preventing, removing or inhibiting is important is that of a ship hull. Biofilms, such as those of Halomonas pacifica, promote the corrosion of the hull of ships and also increase the roughness of the hull, increasing the drag on the ship and thereby increasing fuel costs. The biofilm can also promote the attachment of larger living structures such as barnacles on the ship hull. Fuel can account for half of the cost of marine shipping, and the loss in fuel efficiency due to biofilm formation is substantial.

Substrates on which biofilms can adhere include those of living organisms, as in the case of humans with chronic infections caused by biofilms, as discussed above. Biofilms can also form on the substrates of food contact surfaces, such as those used for processing seafood, and also on food products themselves. Examples of seafood products that may have biofilm contamination include oysters. Human infections caused by the ingestion of raw oysters has been linked to Vibrio vulificus bacterium. Vibrio bacteria attach to algae and plankton in the water and transfer to the oysters and fish that feed on these organisms.

Other examples of substrates or devices on which biofilms can adhere can be found in U.S. Pat. Nos. 5,814,668 and 7,087,661; and U.S. Pat. Application Publication Nos. 2006/0228384 and 2006/0018945, each of which is incorporated herein by reference in its entirety.

In some embodiments, methods of enhancing the effects of a biocide are disclosed, comprising the step of administering a compound described herein in combination with a biocide, the active compound being administered in an amount effective to enhance the effects of the biocide.

“Administering” or “administration of” a compound described herein and/or biocide as used herein in inclusive of contacting, applying, etc. (e.g., contacting with an aqueous solution, contacting with a surface (e.g., a hospital surface such as a table, instrumentation, etc.)), in addition to providing to a subject (for example, to a human subject in need of treatment for a microbial infection).

“Enhancing” the effects of a biocide by administering a compound described herein in combination with the biocide refers to increasing the effectiveness of the biocide, such that the microorganism killing and/or growth inhibition is higher at a certain concentration of the biocide administered in combination with the active compound than without. In some embodiments, a bacteria or other microorganism is “sensitized” to the effects of a biocide, such that the bacteria or other microorganism that was resistant to the biocide prior to administering the compound described herein (e.g., little to none, or less than 20, 10, 5 or 1% are killed upon application) is rendered vulnerable to that biocide upon or after administering the compound (e.g., greater than 20, 30, 40, 50, 60, 70, 80, 90, or 95% or more are killed).

As used herein, the administration of two or more compounds (inclusive of the compounds described herein and biocides) “in combination” means that the two compounds are administered closely enough in time that the administration of or presence of one alters the biological effects of the other. The two compounds may be administered simultaneously (concurrently) or sequentially.

Simultaneous administration of the compounds may be carried out by mixing the compounds prior to administration, or by administering the compounds at the same point in time but at different anatomic sites or using different routes of administration, or administered at times sufficiently close that the results observed are indistinguishable from those achieved when the compounds are administered at the same point in time.

Sequential administration of the compounds may be carried out by administering, e.g., an active compound at some point in time prior to administration of a biocide, such that the prior administration of active compound enhances the effects of the biocide (e.g., percentage of microorganisms killed and/or slowing the growth of microorganisms). In some embodiments, an active compound is administered at some point in time prior to the initial administration of a biocide. Alternatively, the biocide may be administered at some point in time prior to the administration of an active compound, and optionally, administered again at some point in time after the administration of an active compound.

Also provided herein are methods for controlling biofilm formation on a substrate. Methods for controlling biofilm formation on a substrate can comprise contacting the substrate with a compound described herein in an amount effective to inhibit biofilm formation.

The biofilm can comprise Gram-positive bacteria or Gram-negative bacteria. In some embodiments, the biofilm can comprise Gram-positive bacteria. Examples of Gram-positive bacteria affected by compounds described herein include, but are not limited to, bacteria of the genera Listeria, Staphylococcus, Streptococcus, Bacillus, Corynebacterium, Peptostreptococcus, and Clostridium. For example, the bacteria can include Listeria monocytogenes, Staphylococcus aureus, Streptococcus pyogenes, Streptococcus pneumoniae, Bacillus cereus, Bacillus anthracis, Clostridium botulinum, Clostridium perfringens, Clostridium difficile, Clostridium tetani, Corynebacterium diphtheriae, Corynebacteruim ulcerans, and Peptostreptococcus anaerobius. Other examples of Gram-positive bacteria include, for example, bacteria of the genera Actinomyces, Propionibacterium, Nocardia and Streptomyces.

In some embodiments, the biofilm can comprise Gram-negative bacteria. Examples of Gram-positive bacteria affected by compounds described herein include, but are not limited to, bacteria of the genera Escherichia, Salmonella, Vibrio, Helicobacter, Pseudomonas, Bordetella, Vibrio, Haemophilus, Halomonas, and Acinetobacter. For example, the bacteria can include Pseudomonas aeuroginosa, Bordetella pertussis, Vibrio vulnificus, Haemophilus influenzae, Halomonas pacifica, and Acinetobacter baumannii. Other examples of Gram-negative bacteria include, for example, bacteria of the genera Klebsiella, Proteus, Neisseria, Helicobacter, Brucella, Legionella, Campylobacter, Francisella, Pasteurella, Yersinia, Bartonella, Bacteroides, Streptobacillus, Spirillum, Moraxella and Shigella.

Also provided are methods for treating chronic bacterial infections in a subject in need thereof. These methods can comprise administering a compound described herein to a subject in an amount effective to inhibit, reduce, or remove a biofilm component of said chronic bacterial infection. “Treating” as used herein refers to any type of activity that imparts a benefit to a patient afflicted with a disease, including improvement in the condition of the patient (e.g., in one or more symptoms), delay in the progression of the disease, delay in onset of the disease, etc.

A “chronic bacterial infection” is a bacterial infection that is of a long duration or frequent recurrence. For example, a chronic middle ear infection, or otitis media, can occur when the Eustachian tube becomes blocked repeatedly due to allergies, multiple infections, ear trauma, or swelling of the adenoids. The definition of “long duration” will depend upon the particular infection. For example, in the case of a chronic middle ear infection, it may last for weeks to months. Other known chronic bacterial infections include urinary tract infection (most commonly caused by Escherichia coli and/or Staphylococcus saprophyticus), gastritis (most commonly caused by Helicobacter pylori), respiratory infection (such as those commonly afflicting patents with cystic fibrosis, most commonly caused by Pseudomonas aeuroginosa), cystitis (most commonly caused by Escherichia coli), pyelonephritis (most commonly caused by Proteus species, Escherichia coli and/or Pseudomonas species), osteomyelitis (most commonly caused by Staphylococcus aureus, but also by Escherichia coli), bacteremia, skin infection, rosacea, acne, chronic wound infection, infectious kidney stones (can be caused by Proteus mirabilis), bacterial endocarditis, and sinus infection. A common infection afflicting pigs is atrophic rhinitis (caused by Bordatella species, e.g. Bordatella rhinitis).

Also disclosed is a method of clearing a preformed biofilm from a substrate comprising the step of administering an effective amount of a compound described herein to said substrate, wherein said effective amount will reduce the amount of said biofilm on said substrate. “Preformed biofilm” is a biofilm that has begun to adhere to a substrate. The bioflilm may or may not yet be fully formed.

Also provided are methods of treating subjects infected with a bacterium. Methods of treating a subject infected with a bacterium can comprise administering to the subject a therapeutically effective amount of a compound described herein. In some embodiments, the bacterium can comprise a Gram-positive bacterium. Examples of Gram-positive bacteria affected by the compounds described herein include, but are not limited to, bacteria of the genera Listeria, Staphylococcus, Streptococcus, Bacillus, Corynebacterium, Peptostreptococcus, and Clostridium. For example, the bacterium can include Listeria monocytogenes, Staphylococcus aureus, Streptococcus pyogenes, Streptococcus pneumoniae, Bacillus cereus, Bacillus anthracis, Clostridium botulinum, Clostridium perfringens, Clostridium difficile, Clostridium tetani, Corynebacterium diphtheriae, Corynebacteruim ulcerans, and Peptostreptococcus anaerobius. Other examples of Gram-positive bacteria include, for example, bacteria of the genera Actinomyces, Propionibacterium, Nocardia and Streptomyces. In certain embodiments, the bacterium can include Staphylococcus aureus (methicillin sensitive), Staphylococcus aureus (methicillin resistant), Staphylococcus aureus (vancomycin resistant), Streptococcus pneumonia (penicillin sensitive), Streptococcus pneumonia (penicillin resistant), Staphylococcus epidermis (multiple drug resistant), Enterococcus faecalis (vancomycin sensitive), Enterococcus faecium (vancomycin resistant), and/or Haemophilus influenzae.

In some embodiments, the bacterium can comprise Gram-negative bacteria. Examples of Gram-negative bacteria affected by the oxazolidinone derivatives described herein include, but are not limited to, bacteria of the genera Escherichia, Salmonella, Vibrio, Helicobacter, Pseudomonas, Bordetella, Vibrio, Haemophilus, Halomonas, and Acinetobacter. For example, the bacteria can include Pseudomonas aeuroginosa, Bordetella pertussis, Vibrio vulnificus, Haemophilus influenzae, Halomonas pacifica, and Acinetobacter baumannii. Other examples of Gram-negative bacteria include, for example, bacteria of the genera Klebsiella, Proteus, Neisseria, Helicobacter, Brucella, Legionella, Campylobacter, Francisella, Pasteurella, Yersinia, Bartonella, Bacteroides, Streptobacillus, Spirillum, Moraxella and Shigella. In some embodiments, the bacterium can comprise a Gram-negative bacterium. For example, the bacterium can include Salmonella, E. Coli. Acinetobacter baumanii, Pseudomonas aeruginosa or Klebsiella pneumoniae.

By way of non-limiting illustration, examples of certain embodiments of the present disclosure are given below.

### Examples

### Materials and Methods

Tetrahydrofuran (THF) and dichloromethane (DCM) were purified using an alumina filtration system before use. Aldehydes were purchased from a commercial chemical company and used as received unless otherwise noted. Test reactions were monitored by TLC analysis (pre-coated silica gel 60 F₂₅₄ plates, 250 mm layer thickness) and visualization was accomplished with a 254 nm UV light and by staining with a KMnO₄ solution (1.5 g of KMnO₄, 10 g of K₂CO₃, and 1.25 mL of a 10% NaOH solution in 200 mL of water). Test reactions were also monitored by LC-MS (2.6 mm C18 50×2.10 mm column). Yields that are reported are from reactions that were not monitored by TLC or LC-MS. A Biotage® flash chromatography system was used to purify all of the compounds. Melting points were determined using a DigiMelt apparatus. Infrared spectra were determined on a Bruker Alpha spectrometer. ¹H and ¹³C NMR spectra were obtained on a 500, or 600 MHz instrument in CDCl₃ or DMSO-d₆ as indicated. Chemical shifts were reported as observed in parts per million with the residual solvent peak used as an internal standard (CDCl₃=7.26 ppm for ¹H and 77.16 ppm for ¹³C; DMSO-d₆=2.50 ppm for ¹H and 39.52 ppm for ¹³C). ¹H NMR spectra were run at 500 or 600 MHz and are tabulated as follows: chemical shift, multiplicity (s=singlet, d=doublet, t=triplet, q=quartet, p=pentet, m=multiplet, bs=broad singlet, dt=doublet of triplet, tt=triplet of triplet), number of protons, and coupling constant(s). ¹³C NMR spectra were run at 125 or 150 MHz using a proton-decoupled pulse sequence with a d1 of 1 second unless otherwise noted, and are tabulated by observed peak. High resolution mass spectra were obtained on an ion trap mass spectrometer using heated electrospray ionization (HESI).

### Preparation of Vancomycin-2,3-Pyrrolidinediones Conjugates

A series of example vancomycin-2,3-pyrrolidinedione conjugates (Compounds 34-41) were prepared as described below.

General procedure: In a 50 mL RB flask vancomycin hydrochloride (100 mg, 69 μmol) was dissolved in 2.5 mL of dry DMSO and 2.5 mL of dry DMF. The corresponding pyrrolidinone diamine (3.0 equiv) was added and the mixture cooled down to 0° C. in an ice bath. Then, recently distilled DIPEA (over CaH₂) (5.0 equiv) was added, followed by PyBOP (3 equiv). The reaction mixture was stirred at 400 RPM for 20 h total letting the reaction to reach room temperature overnight. The reaction was followed by LCMS, until no more desired product was being formed, and no more vancomycin SM was observed. DMSO and DMF were removed via ultra-high vacuum lyophilization, and the resulting oil was triturated using DCM and the mixture centrifuged at 4700 RPM for 5 minutes, and the remaining white solid was decanted. The product was purified using reverse phase flash chromatography, to yield the desired product.

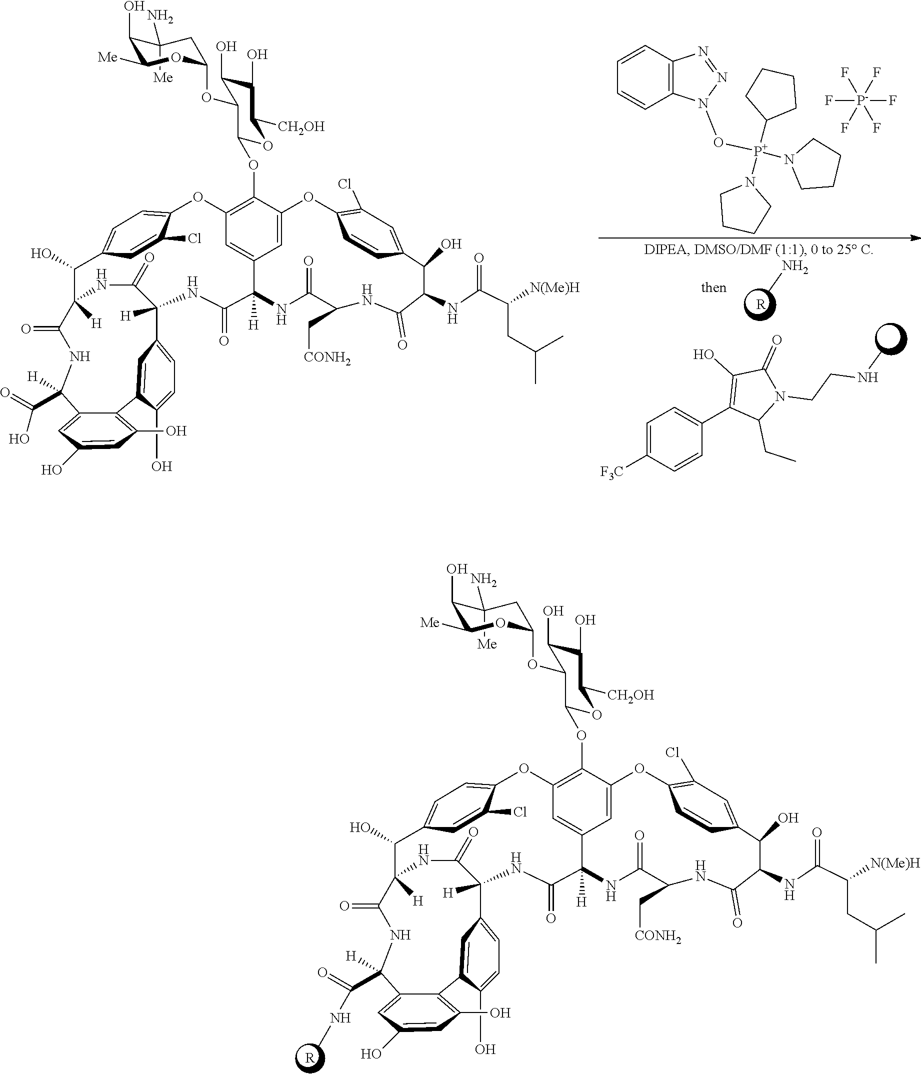

(3S,6R,7R,22R,23S,26S,36R,38aR)-3-(2-Amino-2-oxoethyl)-44-(((2R,3S,4R,6S)-3-(((2S,4S,5S,6S)-4-amino-5-hydroxy-4,6-dimethyletrahydro-2H-pyran-2-yl)oxy)-4,5-dihydroxy-6-(hydroxymethyl)tetrahydro-2H-pyran-2-yl)oxy)-10,43-dichloro-N-(2-(2-ethyl-4-hydroxy-5-oxo-3-(4-(trifluoromethyl)phenyl)-2,5-dihydro-H-pyrrol-1-yl)ethyl)-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclobexadecino[4,5-d][1]oxa[7,17]diazacyclotetracosine-26-carboxamide (1): According to general procedure, 1 was synthesized in a yield of 73 mg (31%) as a white solid: ¹H NMR (500 MHz, DMSO-d₆) δ 10.68 (s, 1H), 10.54 (s, 1H), 10.41 (s, 1H), 9.35 (s, 2H), 8.98 (s, 3H), 8.73 (s, 2H), 8.50 (s, 2H), 8.18 (s, 1H), 7.87 (d, J=8.1 Hz, 2H), 7.78 (dt, J=26.9, 8.5 Hz, 4H), 7.62 (s, 3H), 7.57-7.42 (m, 3H), 7.35 (dd, J=8.3, 4.1 Hz, 1H), 7.21 (t, J=11.2 Hz, 2H), 7.10 (s, 1H), 6.77 (d, J=8.5 Hz, 1H), 6.73-6.63 (m, 2H), 6.38 (d, J=15.7 Hz, 1H), 6.34-6.26 (m, 1H), 5.94 (s, 2H), 5.78 (d, J=7.5 Hz, 1H), 5.62 (s, 1H), 5.27 (dd, J=13.4, 8.6 Hz, 3H), 5.19 (s, 2H), 4.97-4.83 (m, 2H), 4.76 (s, 1H), 4.68 (d, J=6.8 Hz, 1H), 4.46 (s, 2H), 4.38 (d, J=5.4 Hz, 2H), 4.23 (d, J=16.0 Hz, 3H), 3.96 (s, 3H), 3.56 (d, J=10.0 Hz, 3H), 3.44 (s, 3H), 3.27 (d, J=5.7 Hz, 2H), 3.17 (d, J=4.3 Hz, 2H), 3.06 (s, 2H), 2.63 (t, J=4.9 Hz, 2H), 2.15 (d, J=12.7 Hz, 2H), 1.90 (d, J=17.5 Hz, 2H), 1.75-1.62 (m, 3H), 1.56 (d, J=7.7 Hz, 1H), 1.30 (d, J=3.7 Hz, 2H), 1.07 (d, J=6.2 Hz, 2H), 0.92 (d, J=5.9 Hz, 2H), 0.87 (d, J=6.0 Hz, 2H), 0.33 (dp, J=7.5, 4.9, 2.8 Hz, 3H). LRMS (ESI+APCI) m/z calculated for C₈₁H₉₁Cl₂F₃N₁₁O₂₅ [M+H]⁺ 1746.55, found [M+H]^{t} 1746.2, [M−H]^{t} 1743.8.

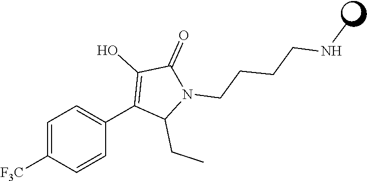 [SMILES: CCC1C(c2ccc(C(F)(F)F)cc2)=C(O)C(=O)N1CCCCNC]

(3S,6R,7R,22R,23S,26S,36R,38aR)-3-(2-Amino-2-oxoethyl)-44-(((2R,3S,4R,6S)-3-(((2S,4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-10,43-dichloro-N-(4-(2-ethyl-4-hydroxy-5-oxo-3-(4-(trifluoromethyl)phenyl)-2,5-dihydro-1H-pyrrol-1-yl)butyl)-7,22,28,30,32-pentahydroxy-6-((R)-4-methyl-2-(methylamino)pentanamido)-2,5,24,38,39-pentaoxo-2,3,4,5,6,7,23,2425,26,36,37,38,38a-tetradecahydro-1H,22H-23,36-(epiminomethano)-8,11:18,21-dietheno-13,16:31,35-di(metheno)benzo[n][1]oxa [6,9]diazacyclohexadecino[4,5-d][1]oxa[7,17]diazacyclotetracosine-26-carboxamide (2): According to general procedure, 2 was synthesized in a yield of 57 mg (23%) as a white solid: ¹H NMR (500 MHz, Methanol-d4) δ 8.99 (s, 2H), 8.59 (s, 2H), 7.85 (d, J=8.4 Hz, 2H), 7.78 (d, J=8.4 Hz, 1H), 7.70 (q, J=6.8 Hz, 4H), 7.63 (d, J=8.6 Hz, 1H), 7.23 (s, 2H), 7.07 (s, 1H), 6.78 (s, 2H), 6.47-6.39 (m, 2H), 5.82 (s, 4H), 5.44 (s, 3H), 5.36 (d, J=14.2 Hz, 2H), 5.30 (s, 2H), 4.77 (t, J=3.4 Hz, 2H), 4.68 (d, J=21.7 Hz, 3H), 4.29 (s, 1H), 4.20 (s, 1H), 4.05 (t, J=7.0 Hz, 1H), 3.98 (s, 1H), 3.86-3.81 (m, 2H), 3.61 (t, J=8.9 Hz, 2H), 3.52 (s, 2H), 3.37 (s, 2H), 3.17 (dd, J=3.3, 1.7 Hz, 3H), 3.04-2.96 (m, 3H), 2.92 (d, J=17.4 Hz, 2H), 2.76 (s, 2H), 2.66 (s, 1H), 2.10-1.98 (m, 4H), 1.92 (d, J=13.4 Hz, 2H), 1.87 (ddd, J=13.1, 8.1, 4.3 Hz, 3H), 1.71 (dq, J=31.0, 7.3 Hz, 7H), 1.60 (d, J=7.6 Hz, 3H), 1.51 (s, 3H), 1.29 (s, 1H), 1.20 (d, J=6.4 Hz, 2H), 0.98 (d, J=17.5 Hz, 5H), 0.45 (t, J=7.4 Hz, 3H), 0.41 (t, J=3.8 Hz, 2H). LRMS (ESI+APCI) m/z calculated for C₈₃H₉₅Cl₂F₃N₁₁O₂₅[M+H]⁺ 1774.61, found [M+H]⁺ 1774.8, [M−H]⁺ 1772.4, [M+2H]²⁺ 887.6.

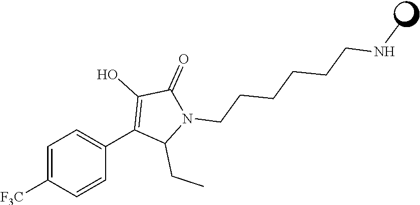 [SMILES: CCC1C(c2ccc(C(F)(F)F)cc2)=C(O)C(=O)N1CCCCCCNC]

(3S,6R,7R,22R,23S,26S,36R,38aR)-3-(2-Amino-2-oxoethyl)-44-(((2R,3S,4R,6S)-3-(((2S,4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-10,43-dichloro-N-(6-(2-ethyl-4-hydroxy-5-oxo-3-(4-(trifluoromethyl)phenyl)-2,5-dihydro-1H-pyrrol-1-yl)hexyl)-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa [6,9]diazacyclohexadecino[4,5-d][1]oxa[7,17]diazacyclotetracosine-26-carboxamide (3): According to general procedure, 3 was synthesized in a yield of 68 mg (32%) as a white solid: ¹H NMR (500 MHz, Methanol-d4) δ 8.98 (s, 2H), 8.57 (s, 2H), 7.70 (d, J=8.1 Hz, 4H), 7.62 (d, J=8.7 Hz, 3H), 7.34 (s, 3H), 7.21 (s, 2H), 7.06 (s, 1H), 6.79 (s, 3H), 6.43 (d, J=2.3 Hz, 1H), 6.40 (s, 1H), 5.86 (s, 4H), 5.47 (d, J=20.7 Hz, 3H), 5.36 (d, J=14.7 Hz, 2H), 5.29 (s, 1H), 4.79-4.73 (m, 2H), 4.71 (s, 1H), 4.65 (s, 1H), 4.29 (s, 2H), 4.19 (s, 1H), 4.05 (t, J=6.9 Hz, 2H), 3.98 (s, 1H), 3.90 (s, 2H), 3.86-3.75 (m, 4H), 3.62 (t, J=9.0 Hz, 2H), 3.57 (s, 2H), 3.47-3.43 (m, 1H), 3.36 (d, J=8.0 Hz, 2H), 3.19-3.10 (m, 2H), 2.96-2.89 (m, 2H), 2.77 (s, 3H), 2.66 (s, 1H), 2.08 (d, J=4.7 Hz, 1H), 1.93 (d, J=13.3 Hz, 2H), 1.87 (d, J=6.9 Hz, 3H), 1.77-1.64 (m, 5H), 1.59 (d, J=6.2 Hz, 3H), 1.52 (s, 3H), 1.48-1.35 (m, 5H), 1.20 (d, J=6.4 Hz, 3H), 0.97 (d, J=19.1 Hz, 6H), 0.44 (t, J=7.3 Hz, 3H). LRMS (ESI+APCI) m/z calculated for C₈₅H₉₉Cl₂F₃N₁₁O₂₅ [M+H]⁺ 1802.66, found [M+H]⁺ 1802.3. [M−H]⁺ 1800.5.

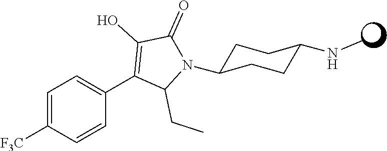 [SMILES: CCC1C(c2ccc(C(F)(F)F)cc2)=C(O)C(=O)N1C1CCC(NC)CC1]

(3S,6R,7R,22R,23S,26S,36R,3aR)-3-(2-Amino-2-oxoethyl)-44-(((2R,3S,4R,6S)-3-(((2S,4S,%6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-10,43-dichloro-N-((1r,4S)-4-(2-ethyl-4-hydroxy-5-oxo-3-(4-(trifluoromethyl)phenyl)-2,5-dihydro-1H-pyrrol-1-yl)cyclohexyl)-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclohexadecino[4,5-d][1]oxa[7,17]diazacyclotetracosine-26-carboxamide (4): According to general procedure, 4 was synthesized in a yield of 81 mg (65%) as a white solid: ¹H NMR (500 MHz, DMSO-d₆) δ 10.43 (s, 2H), 9.31 (s, 2H), 8.97 (s, 2H), 8.89 (s, 2H), 8.69 (s, 2H), 8.48 (s, 3H), 7.88 (s, 1H), 7.84-7.71 (m, 5H), 7.61 (s, 2H), 7.57-7.42 (m, 3H), 7.34 (d, J=8.2 Hz, 1H), 7.20 (t, J=7.0 Hz, 2H), 7.08 (s, 1H), 6.76 (d, J=8.5 Hz, 1H), 6.72-6.60 (m, 2H), 6.50 (s, 2H), 6.34 (d, J=11.8 Hz, 2H), 5.93 (s, 2H), 5.77 (d, J=7.9 Hz, 1H), 5.62 (s, 1H), 5.44 (s, 2H), 5.25 (d, J=9.3 Hz, 2H), 5.19 (d, J=4.9 Hz, 3H), 4.92 (s, 1H), 4.80 (d, J=10.2 Hz, 1H), 4.72-4.63 (m, 1H), 4.46 (s, 2H), 4.20 (s, 3H), 3.96 (s, 2H), 3.27 (s, 2H), 3.17 (d, J=3.8 Hz, 2H), 3.06 (d, J=30.6 Hz, 2H), 2.64 (t, J=5.3 Hz, 3H), 2.16 (s, 2H), 2.00 (d, J=11.2 Hz, 2H), 1.89 (s, 3H), 1.81 (s, 2H), 1.77-1.63 (m, 4H), 1.56 (s, 1H), 1.49-1.37 (m, 2H), 1.30 (s, 3H), 1.07 (d, J=6.2 Hz, 2H), 0.89 (dd, J=24.7, 6.0 Hz, 5H), 0.39 (q, J=7.8 Hz, 3H). LRMS (ESI+APCI) m/z calculated for C₈₅H₉₇Cl₂F₃N₁₁O₂₅ [M+H]⁺ 1801.64, found [M+2H]²⁺ 901.3, [M−2H]²⁺−898.7, [M+2H+Na]³⁺ 608.4, [M+3H]³⁺ 601.8.

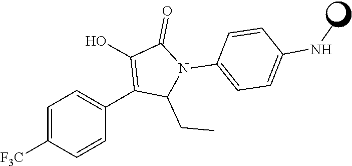 [SMILES: CCC1C(c2ccc(C(F)(F)F)cc2)=C(O)C(=O)N1c1ccc(NC)cc1]

(3S,6R,7R,22R,23S,26S,36R,38aR-(2-Amino-2-oxoethyl)-44-(((2R,3S,4R,6S)-3-(((2S4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-10,43-dichloro-N-(4-(2-ethyl-4-hydroxy-5-oxo-3-(4-(trifluoromethyl)phenyl)-2,5-dihydro-1H-pyrrol-1-yl)phenyl)-7,22,28,30,32-pentahydroxy-6-((R)-4-methyl-2-(methylamino) pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclohexadecino[4,5-d][1]oxa[7,17]diazacyclotetracosine-26-carboxamide (5): According to general procedure, 5 was synthesized in a yield of 6 mg (5%) as a brown solid: Further spectral analysis is needed. LRMS (ESI+APCI) m/z calculated for C₈₅H₉₁Cl₂F₃N₁₁O₂₅ [M+H]⁺ 1794.60, found [M+H]⁺ 1794.09, [M+2ACN+H]⁺ 1876.66.

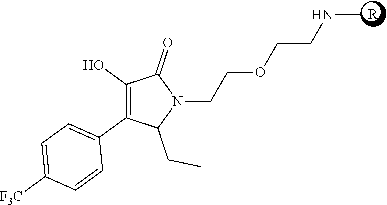

(3S,6R,7R,22R,23S,26S,36R,R)-3-(2-amino-2-oxoethyl)-44-(((2S,3R,4S,5S,6R)-3-(((2S,4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-19,45-dichloro-N-(2-(2-(2-ethyl-4-hydroxy-5-oxo-3-(4-(trifluoromethyl)phenyl)-2,5-dihydro-1H-pyrrol-1-yl)ethoxy)ethyl)-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclohexadecino[4,5-d][1]oxa[7,17]diazacyclotetracosine-26-carboxamide (6): According to general procedure, 6 was synthetized in a yield of 15 mg (18%) as a white solid. Further spectral analysis is needed. LRMS (ESI+APCI) m/z calculated for C₈₃H₉₅Cl₂F₃N₁₁O₂₆ [M+H]⁺ 1790.6, found [M+2H]²⁺895.6, [M−2H]²⁻894.2.

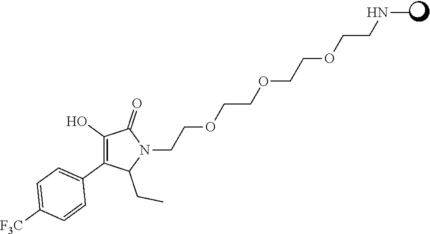 [SMILES: CCC1C(c2ccc(C(F)(F)F)cc2)=C(O)C(=O)N1CCOCCOCCOCCNC]

(3S,6R,7R,22R,23S,26S,36R,38aR)-3-(2-Amino-2-oxoethyl)-44-(((2R,3S,4R,6S)-3-(((2S4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-10,43-dichloro-N-(2-(2-(2-(2-(2-ethyl-4-hydroxy-5-oxo-3-(4-(trifluoromethyl)phenyl)-2,5-dihydro-1H-pyrrol-1-yl)ethoxy)ethoxy)ethoxy)ethyl)-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclohexadecino[4,5-d][1]oxa[7,17]diazacyclotetracosine-26-carboxamide (7): According to general procedure, 7 was synthesized in a yield of 84 mg (54%) as a white solid: ¹H NMR (500 MHz, Methanol-d₄) δ 8.99 (s, 2H), 8.59 (s, 2H), 8.19-8.03 (m, 2H), 7.82 (t, J=8.8 Hz, 2H), 7.73-7.57 (m, 7H), 7.19 (s, 2H), 7.08 (d, J=9.1 Hz, 1H), 6.75 (s, 4H), 6.47-6.35 (m, 3H), 5.96 (s, 3H), 5.50 (d, J=29.6 Hz, 4H), 5.43-5.32 (m, 3H), 5.28 (s, 2H), 4.74 (s, 2H), 4.65 (s, 2H), 4.27 (s, 2H), 4.20 (s, 2H), 4.04 (q, J=6.1, 5.2 Hz, 2H), 3.98 (s, 2H), 3.72 (d, J=4.5 Hz, 3H), 3.68-3.52 (m, 15H), 2.93 (d, J=15.5 Hz, 2H), 2.77 (s, 4H), 2.14-2.01 (m, 4H), 1.93 (d, J=13.4 Hz, 2H), 1.85 (q, J=8.7, 8.3 Hz, 4H), 1.65 (d, J=14.3 Hz, 3H), 1.54 (s, 4H), 1.18 (d, J=6.3 Hz, 3H), 0.94 (d, J=15.1 Hz, 6H), 0.42 (t, J=7.0 Hz, 3H). LRMS (ESI+APCl) r/z calculated for C₈₇H₁₀₃Cl₂F₃N₁₁O₂₈ [M+H]⁺ 1878.71, found [M+H]⁺ 1878.5, [M−H]⁺ 1876.6.

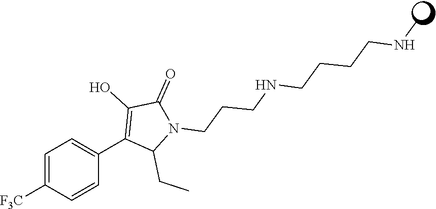 [SMILES: CCC1C(c2ccc(C(F)(F)F)cc2)=C(O)C(=O)N1CCCNCCCCNC]

(3S,6R,7R,22R,23S,2,36R,38aR)-3-(2-Amino-2-oxoethyl)-44-(((2R,3S,4R,6S)-3-(((2S,4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-10,43-dichloro-N-(4-((3-(2-ethyl-4-hydroxy-5-oxo-3-(4-(trifluoromethyl)phenyl)-2,5-dihydro-1H-pyrrol-1-yl)propyl)amino)butyl)-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclohexadecino[4,5-d][1]oxa[7,17]diazacyclotetracosine-26-carboxamide (8): According to general procedure, 8 was synthesized in a yield of 29.7 mg (12%) as a white or light brown solid: ¹H NMR (500 MHz, Methanol-d₄) δ 9.01 (s, 2H), 8.71 (s, 1H), 8.25 (s, 1H), 8.12-8.04 (m, 1H), 7.89-7.82 (m, 2H), 7.73-7.68 (m, 3H), 7.68-7.61 (m, 2H), 7.25 (d, J=8.4 Hz, 1H), 7.11 (s, 1H), 7.01 (s, 1H), 6.85 -6.77 (m, 1H), 6.47-6.42 (m, 1H), 6.37 (dd, J=11.7, 2.4 Hz, 1H), 5.80 (s, 2H), 5.46 (d, J=7.8 Hz, 1H), 5.44-5.37 (m, 2H), 5.32 (d, J=15.6 Hz, 2H), 5.00 (s, 1H), 4.81-4.75 (m, 2H), 4.70 (s, 1H), 4.65-4.55 (m, 2H), 4.31 (s, 1H), 4.26 (s, 1H), 4.07 (t, J=7.1 Hz, 1H), 3.98 (s, 1H), 3.91-3.70 (m, 5H), 3.61 (s, 2H), 3.51 (t, J=9.2 Hz, 2H), 3.47-3.34 (m, 4H), 3.33 (s, 1H), 3.27-3.20 (m, 2H), 3.18-2.95 (m, 1OH), 2.94-2.86 (m, 2H), 2.76 (s, 2H), 2.26 (s, 2H), 2.12-1.98 (m, 5H), 1.93 (d, J=13.0 Hz, 3H), 1.88-1.61 (m, 11H), 1.50 (s, 2H), 1.29 (s, 1H), 1.20 (d, J=6.3 Hz, 2H), 1.01 (d, J=6.3 Hz, 2H), 0.97 (d, J=6.4 Hz, 2H), 0.90 (s, 1H), 0.84-0.72 (m, 2H), 0.49-0.42 (m, 3H). LRMS (ESI+APCI) m/z calculated for C₈₆H₁₀₂Cl₂F₃N₁₂O₂₃ [M+H]⁺ 1831.70, found [M+H]⁺ 1831.1, [M−H]⁺ 1829.6, [M+2H]²⁺916.2.

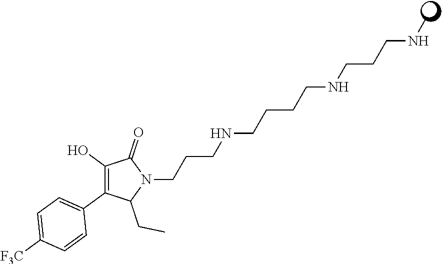 [SMILES: CCC1C(c2ccc(C(F)(F)F)cc2)=C(O)C(=O)N1CCCNCCCCNCCCNC]

(3S,6R,7R,22R,23S,26S,36R,3aR)-3-(2-Amino-2-oxoethyl)-44-(((2R,3S,4R,6S)-3-(((2S,4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-10,43-dichloro-N-(3-((4-((3-(2-ethyl-4-hydroxy-5-oxo-3-(4-(trifluoromethyl)phenyl)-2,5-dihydro-1H-pyrrol-1-yl)propyl)amino)butyl)amino)propyl)-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclohexadecino[4,5-d][1]oxa[7,17]diazacyclotetracosine-26-carboxamide (9b): According to general procedure, 9b was synthesized in a yield of 18 mg (14%) as a white or light brown solid: ¹H NMR (500 MHz, Methanol-d₄) δ 9.03 (s, 1H), 8.91 (s, 1H), 8.77 (d, J=29.2 Hz, 1H), 8.17-7.97 (m, 2H), 7.87 (dd, J=16.9, 8.3 Hz, 3H), 7.72 (dd, J=21.7, 8.2 Hz, 4H), 7.66-7.54 (m, 3H), 7.50 (q, J=7.4, 6.9 Hz, 1H), 7.41 (d, J=8.0 Hz, 1H), 7.28 (d, J=8.1 Hz, 1H), 6.88 (d, J=78.0 Hz, 3H), 6.43 (d, J=21.3 Hz, 1H), 6.34 (s, 1H), 5.89 (d, J=65.7 Hz, 3H), 5.38 (td, J=43.4, 41.1, 18.5 Hz, 4H), 4.80 (s, 3H), 4.72 (s, 1H), 4.59 (s, 1H), 4.30 (d, J=44.2 Hz, 2H), 4.08 (s, 1H), 3.83 (dq, J=14.4, 7.0, 5.7 Hz, 3H), 3.64 (s, 1H), 3.46-3.34 (m, 5H), 3.13 (t, J=7.9 Hz, 4H), 3.06 (p, J=11.8, 9.0 Hz, 12H), 2.77 (s, 2H), 2.66 (s, 3H), 2.07 (tt, J=14.5, 7.3 Hz, 8H), 1.98-1.71 (m, 13H), 1.65 (s, 3H), 1.52 (d, J=21.6 Hz, 2H), 1.37 (dd, J=6.8, 4.2 Hz, 1H), 1.29 (s, 1H), 1.19 (s, 2H), 0.94 (t, J=20.2 Hz, 4H), 0.45 (t, J=7.3 Hz, 3H). LRMS (ESI+APCI) m/z calculated for C₈₉H₁₀₉Cl₂F₃N₁₃O₂₅ [M+H]⁺ 1888.80, found [M+H]⁺ 1888.4, [M−H]⁺ 1886.7.

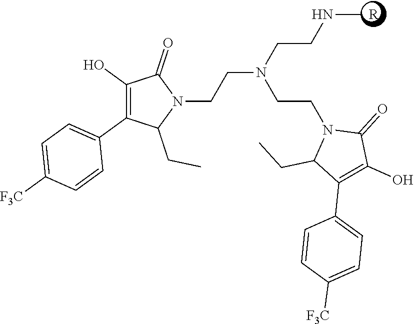

(3S,6R,7R,22R,23S,26S,36R,38aR)-3-(2-amino-2-oxoethyl)-44-(((2S,3R,4S,5S,6R)-3-(((2S,4S,5S,6S)-4-amino-5-hydroxy-4,6-dimethyltetrahydro-2H-pyran-2-yl)oxy)-4,5-dihydroxy-6-(hydroxymethyl)tetrahydro-2H-pyran-2-yl)oxy)-N-(2-(bis(2-(2-ethyl-4-hydroxy-5-oxo-3-(4-(trifluoromethyl)phenyl)-2,5-dihydro-1H-pyrrol-1-yl)ethyl)amino)ethyl)-19,45-dichloro-7,22,28,30,32-pentahydroxy-6-((R)-4-methyl-2-(methylamino)pentanamido)-2,5,24,38,39-pentaoxo-2,3,4,5,6,7,23,24,25,26,36,37,38,38a-tetradecahydro-1H,22H-23,36-(epiminomethano)-8,11:18,21-dietheno-13,16:31,35-di(metheno)benzo[n][1]oxa[6,9]diazacyclohexadecino[4,5-d][1]oxa[7,17]diazacyclotetracosine-26-carboxamide (10) According to general procedure, 6 was synthetized in a yield of 9 mg (25%) as a light tan solid. ¹H NMR (500 MHz, DMSO-d₆) δ 8.58 (s, 1H), 8.47 (s, 1H), 8.34 (s, 4H), 8.26-8.13 (m, 1H), 7.99 (s, 1H), 7.88 (s, 1H), 7.82-7.71 (m, 4H), 7.68-7.62 (m, 2H), 7.48 (dd, J=21.8, 8.4 Hz, 3H), 7.39-7.28 (m, 3H), 7.27-7.19 (m, 3H), 7.19-7.10 (m, 2H), 6.86 (s, 1H), 6.79-6.62 (m, 3H), 6.62-6.49 (m, 1H), 6.36 (s, 1H), 6.27 (s, 1H), 5.78-5.71 (m, 1H), 5.53 (s, 1H), 5.36 (s, 1H), 5.27 (d, J=7.5 Hz, 1H), 5.22 (d, J=4.1 Hz, 2H), 5.15 (d, J=4.3 Hz, 1H), 4.88 (s, 1H), 4.80-4.60 (m, 3H), 4.50-4.33 (m, 3H), 4.23 (d, J=11.6 Hz, 1H), 3.80 (s, 2H), 3.67 (d, J=10.8 Hz, 2H), 3.60-3.40 (m, 5H), 3.35-3.22 (m, 4H), 3.16-3.02 (m, 5H), 2.80-2.65 (m, 6H), 2.30 (d, J=4.1 Hz, 6H), 2.18-2.10 (m, 2H), 2.08 (s, 1H), 1.78-1.57 (m, 6H), 1.48 (dt, J=13.7, 6.8 Hz, 2H), 1.40 (dt, J=13.9, 7.2 Hz, 2H), 1.32-1.21 (m, 5H), 1.05 (d, J=6.2 Hz, 3H), 0.89 (d, J=6.5 Hz, 3H), 0.85 (d, J=6.4 Hz, 3H), 0.37-0.20 (m, 6H). LRMS (ESI+APCI) m/z calculated for C₉₉H₁₁₀Cl₂F₆N₁₃O₂₇ [M+H]⁺ 2086.8, found [M+2H]²⁺ 1044.3, [M−2H]²⁻ 1041.8, [M+H+2Na]³⁺ 710.8.

### Biological Characterization of Compounds 1-10

General information—Biological Assays. Methicillin-resistant and methicillin sensitive Staphylococcus aureus (MRSA and MSSA respectively) strains were obtained from the Laboratory of Professor Christian Melander (NCSU) (ATCC BAA 44 and 33591) and Dr. Jessica Gilbertie (ATCC 25923). Bacteria were kept in frozen stocks on glycerol at −80° C. until use. Bacteria was streaked onto tryptic-soy agar for colony isolation. Mueller-Hinton broth (MHB, 211443-BD), tryptic soy broth (TSB, Remel: R455052) and D-glucose (CAS: 492-62-6) were purchased from Fisher Scientific. Tryptic soy agar (TSA, cat. #22091) and Linezolid (cat. #P70014) were purchased from Sigma-Aldrich. Bacteria for biofilm inhibition were cultured overnight in TSB-G (tryptic soy broth with 0.5% glucose supplement) in 96 well plates. All assays were run in duplicate and repeated at least two separate times for MIC assays and at least three separate times for biofilm inhibition. All compounds were dissolved in molecular biology grade DMSO as 10 mM stock solutions. Optical densities were measured using a Thermo Scientific Genesys 20 spectrophotometer. Data for biofilm inhibition were collected using a BioTek ELx808 Microplate Reader. All graphs were generated and analyzed using GraphPad Prism 7.

Broth microdilution method for determination of minimum inhibitory concentrations. As prescribed by the Clinical and Laboratory Standards Institute (CLSI) M07-A8, Vol. 29 (2) MSSA (ATCC 25923) and MRSA (ATCC BAA 44 and 33591) was grown in MHB for 6-8 h; this culture was used to inoculate fresh MHB (5×105 CFU/mL). The resulting bacterial suspension was aliquoted (1 mL) into 1.5 mL Eppendorf tubes and compound was added from a 10 mM DMSO stock to achieve the desired initial starting concentration (typically 128 μg/mL). Linezolid (from a 10 mM DMSO stock) was used as a positive control. Inoculated media not treated with compound served as the negative control. The MIC was determined by microbroth dilution following the CLSI guidelines. The MIC was defined as the lowest concentration of antibiotic with no visible growth. The plate was sealed and incubated under stationary conditions at 37° C. After 16 h, MIC values were recorded as the lowest concentration of compound at which no visible growth of bacteria was observed.

Determination of the inhibitory effect of test compounds on MRSA biofilm formation. Inhibition assays were performed using a procedure adapted from S. A. Rogers and C. Melander, Angew. Chem. Int. Ed., 2008, 47, 5229-5231, which is hereby incorporated herein by reference. Briefly, the inhibition assays were performed by subculturing an overnight culture of MRSA (ATCC BAA 44) to an OD600 of 0.01 in TSB-G (tryptic soy broth with a 0.5% glucose supplement). Stock solutions of predetermined concentrations of the test compound were then made using the inoculated TSB-G. These stock solutions were aliquoted (100 μL) into the wells of the 96-well microtiter plate. Sample plates were sealed then incubated for 24 h at 37° C. After incubation, the medium was discarded from the wells and the plates were washed 2× with PBS. Prior to staining, plates were left to dry at ambient temperature for 2-3 h. Plates were then stained with 0.1% solution of crystal violet (CV, 125 IL) and then incubated at ambient temperature for 30 min. Plates were washed with PBS again and the remaining stain was solubilized with 99% ethanol (200 μL). A sample of solubilized CV stain (110 μL) from each well was transferred to the corresponding wells of a polystyrene microtiter dish. Biofilm biomass was quantified by measuring the OD540 of each well and inhibition was calculated as a percentage of the control (no compound); a negative control lane wherein no biofilm was formed served as a background and was subtracted out. Percent inhibition was then plotted against concentration in Prism 7. Each of the four experiments were plotted separately and S15 analyzed by a normalized nonlinear regression. The graphs on the following pages were generated from an average of the total data set. Biofilm data represent four separate experiments, with each experiment performed in duplicate (average of 8 data points for each concentration tested, unless otherwise noted).

Determination of the Minimum Biofilm Eradication Concentrations (MBEC) using the Calgary Biofilm Device (CBD) on MSSA (ATCC 25923) biofilms. MBEC concentrations were measured using a procedure adapted from H. Ceri, et al., J. Clin. Microbiol., 1999, 1771-1776, which is hereby incorporated herein by reference. Briefly, biofilm eradication experiments were performed using MSSA (ATCC 25923) and the Calgary Biofilm Device (CBD) to determine MBEC values for various compounds of interest (Innovotech, product code: 19111). The Calgary device is a 96-well plate with a lid containing 96 pegs that sit in the media contained in the bottom well. Biofilm are established on the individual pegs. The established biofilm (contained on the individual peg) can then be transferred to a new base well for MBEC testing. For the MBEC assay, an overnight culture of MSSA (ATCC 25923) was adjusted to 0.5 McFarland in MHB-G. The CBD was inoculated with 100 μL of the 0.5 McFarland and incubated at 37° C. for 24 hours to establish biofilms. The CBD lid containing the established biofilms on individual pegs was removed, washed 3× with PBS and transferred to another 96-well plate containing serial dilutions of the test compounds (the “challenge plate”) and incubated at 37° C. for 24 hours. The CBD lid was then removed from the challenge plate, washed 3× with PBS to remove any residual compound and placed into a new 96-well base plate containing fresh MHB. The plate was then sonicated for sonicated for 30 minutes to disperse biofilms on S16 the pegs into the fresh MHB in the base well. After sonication, the plate was incubated for 24 hours at 37° C. MBEC values were determined as the lowest test concentration that resulted in no growth in the sonicate fluid.

### Discussion

The biological activity of the Compounds 1-10 was investigated. Table 1 includes a summary of the biological activity of Compounds 1-10. As shown in Table 1, vancomycin-2,3-pyrrolidinedione conjugates generally retained the original antimicrobial activity displayed by vancomycin or improved upon it. Happily, the conjugates did not display reduced water solubility as compared to vancomycin. Rather, the conjugates displayed similar water solubility as compared to vancomycin.

TABLE 1
Summary of the Biological Activity of Vancomycin-
2-3-pyrrolidinedione conjugates (Compounds 1-10).

| Compound | Vanco | 1 | 2 | 3 | 4 | 5 | 6 | 7 | 8 | 9a | 9b | 10 |
|---|---|---|---|---|---|---|---|---|---|---|---|---|
| MIC | 2 | 1 | 2 | 2 | 0.5 | 2 | 1 | 2 | 0.25 | 0.5-1 | 0.5-1 | 16 |
| (S. aureus 25923) | | | | | | | | | | | | |
| (μg/mL) | | | | | | | | | | | | |
| MIC | 1 | 1 | 1 | 2 | 0.5-1 | 1 | 1 | 1 | 0.25-0.5 | 1 | 1 | 8 |
| (S. aureus 29213) | | | | | | | | | | | | |
| (μg/mL) | | | | | | | | | | | | |
| MIC | 2 | 1 | 2 | 2 | 1 | 1 | — | 1 | 0.5 | 1 | 1 | 8 |
| (MRSA 33591) | | | | | | | | | | | | |
| (μg/mL) | | | | | | | | | | | | |
| MBEC | 2048 | 128 | 64 | 64 | 16 | 16 | 32-64 | 128 | 16 | 32 | 32 | — |
| (S. aureus 25923) | | | | | | | | | | | | |
| (μg/mL) | | | | | | | | | | | | |
| MBEC/MIC ratio | 1024 | 128 | 32 | 32 | 32 | 8 | 32-64 | 64 | 64 | 32-64 | 32-64 | 16 |

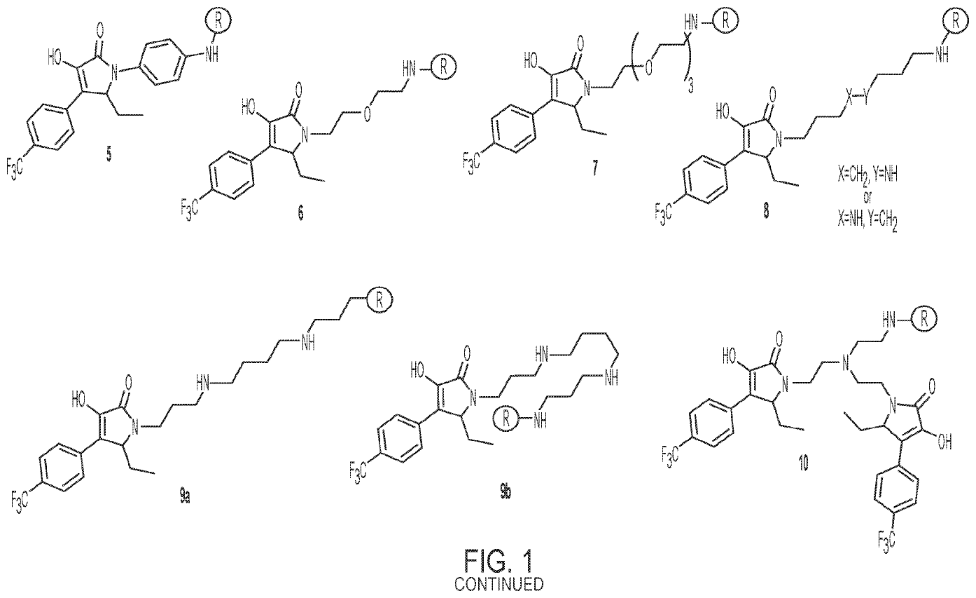

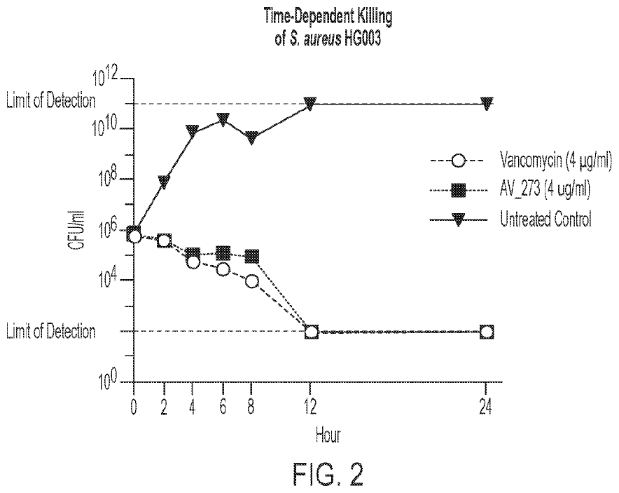

With these initial results in hand, the time-dependent killing of bacterial strains by the vancomycin-2-3-pyrrolidinedione conjugates was investigated. FIG. 2 is a plot showing the time-dependent killing of S. aureus (strain HG003) by vancomycin and an example vancomycin-2-3-pyrrolidinedione conjugate (Compound 4, also referred to as AV-273). FIG. 3 is a plot showing the time-dependent killing of MRSA (strain LAC) by vancomycin and an example vancomycin-2-3-pyrrolidinedione conjugate (Compound 4). In both cases, enumeration of viable bacterial cells in a liquid culture after a single inoculation of a vancomycin-2,3-pyrrolidinedione revealed a time-dependent killing profile that roughly matched vancomycin at the same concentration.

The activity of vancomycin-2-3-pyrrolidinedione conjugates against vancomycin-resistant bacterial strains. As shown in Table 2 below, vancomycin-2,3-pyrrolidinedione conjugates rescued antimicrobial activity against nosocomial isolated vancomycin-resistant Staphylococcus aureus (VRSA), with these conjugates showing the same level of activity (or better) against VRSA as vancomycin does against vancomycin-sensitive pathogens.

TABLE 2
Restoration of vancomycin activity against
vancomycin-resistant bacteria strains.

| Compound | Vanco | 4 | 7 | 8 |
|---|---|---|---|---|
| MIC (VRSA) μg/mL | 64 | 1 | 4 | 2 |
| MIC (vanA VRE 700221) μg/mL | 512 | 8 | — | — |
| MIC (vanB VRE 51299) μg/mL | 256 | 2 | — | — |
| MIC (vanC-1 VRE 49608) μg/mL | 32 | 0.5 | — | — |
| MIC (VSE 2127) μg/mL | 2 | 0.5 | — | — |

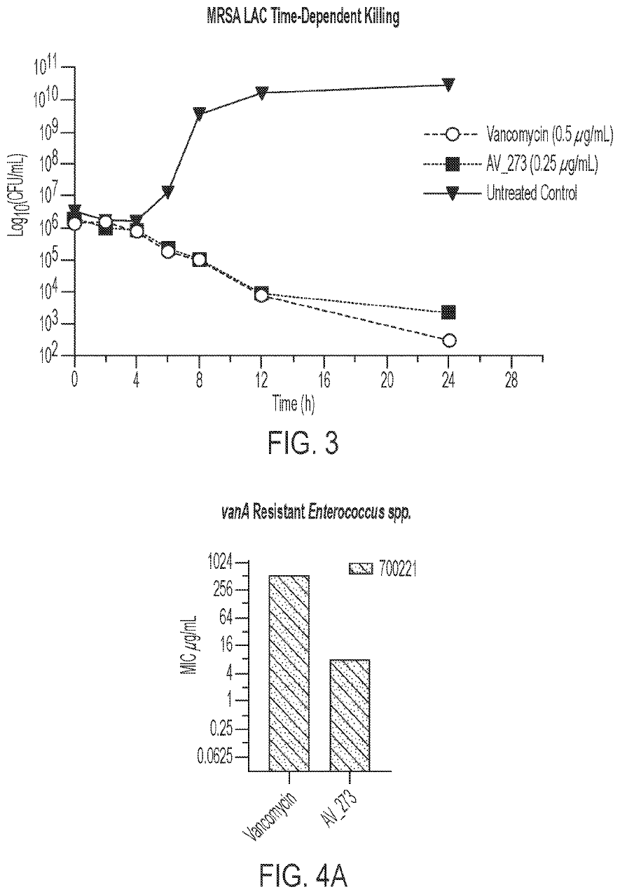

FIG. 4A compares the activity of vancomycin and Compound 4 against vanA-resistant Enterococcus spp. FIG. 4B compares the activity of vancomycin and Compound 4 against vanB-resistant Enterococcus spp. FIG. 4C compares the activity of vancomycin and Compound 4 against vanC1-resistant Enterococcus spp. FIG. 4D compares the activity of vancomycin and Compound 4 against vancomycin-susceptible Enterococcus spp. As shown in FIGS. 4A-4D, an example vancomycin-2,3-pyrrolidinedione conjugate (Compound 4) displays superior in vitro activity against strictly vancomycin-resistant Enterococcus spp. (VRE).

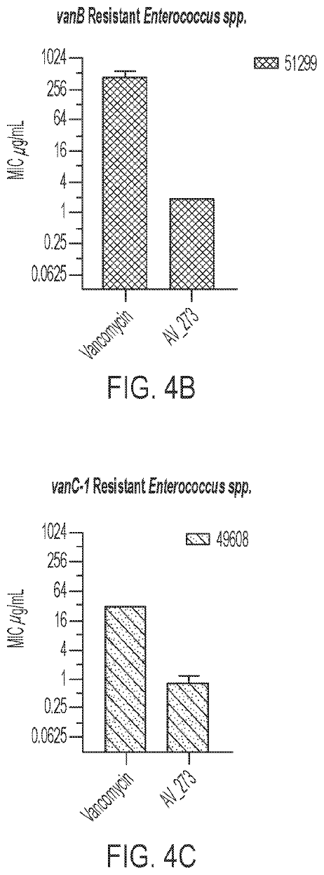

Vancomycin-2,3-pyrrolidinedione conjugates also displayed potent antibiofilm activity against the human pathogen. FIG. 5 is a plot showing the effect of a single dose of different concentrations of Compound 4 on the concentration of bacteria in the supernatant over a methicillin-susceptible Staphylococcus aureus (MSSA ATCC 25923). As shown in FIG. 5, no viable bacterial cells were detected in the treatment supernatant when a single dose of at least 16 μg/mL (which is also the MBEC) of Compound 4 was used (10² was the detection limit of this assay).

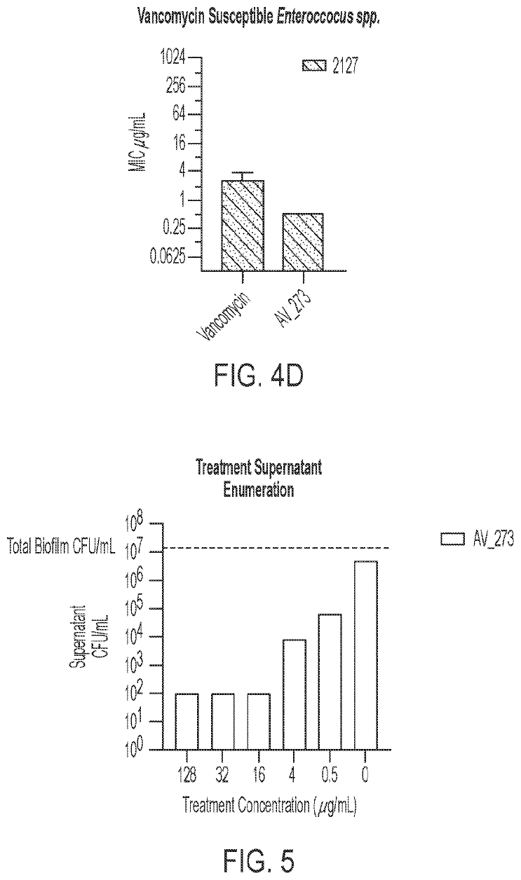

FIG. 6 is a plot showing the time-dependent biofilm population following treatment of a methicillin-susceptible Staphylococcus aureus (MSSA ATCC 25923) biofilm with Compound 4. As shown in FIG. 6, no viable biofilm-embedded bacterial cells were detected at 24 hours elapsed from a single treatment with Compound 4 at twice the MBEC.

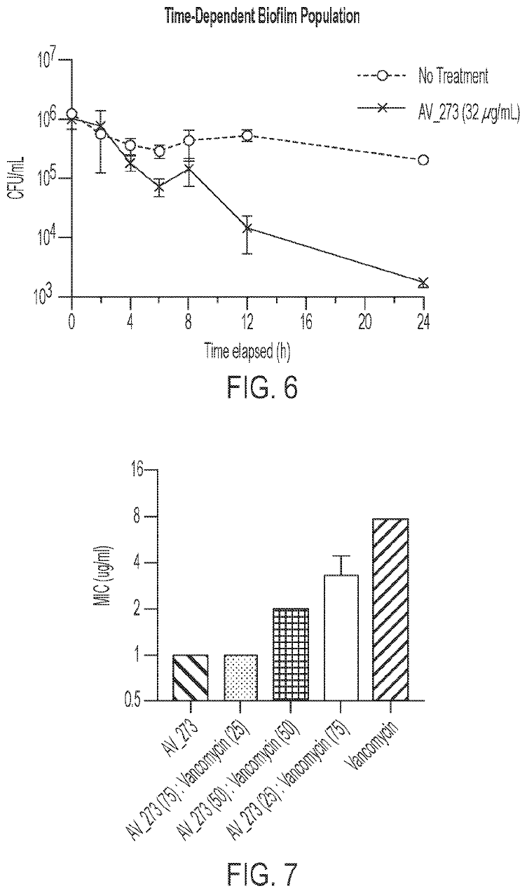

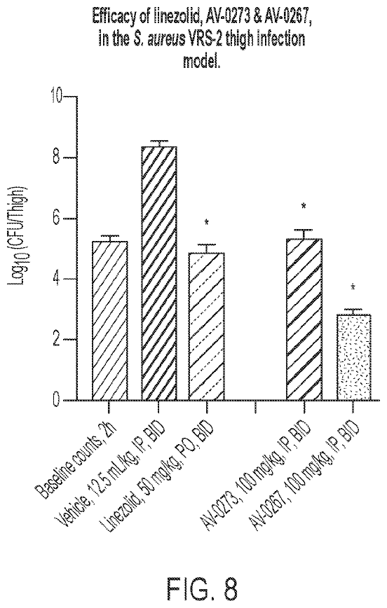

FIG. 7 is a plot showing the MIC (in μg/mL) of Compound 4, vancomycin, and varying ratios of Compound 4 and vancomycin. These results suggested that vancomycin and vancomycin-2,3-pyrrolidinediones conjugates are not synergistic between each other. Further, these results demonstrated that the antibiofilm efficacy of vancomycin-2,3-pyrrolidinediones conjugates depended on the concentration of vancomycin-2,3-pyrrolidinediones conjugate, not the amount of vancomycin present. These results suggested that the conjugate itself is the active species.

### Additional Antimicrobial Assessment of Vancomycin-2,3-Pyrrolidinediones Conjugates

| | | MIC (μg/ml) | MIC (μg/ml) |
|---|---|---|---|
| | | Conjugate 4 | Conjugate 8 |
| Strain/Compound | Vanco | (AV-0273) | (AV-0267) |
| VRS-1 (VRSA) | >64 | 16 | 32 |
| VRS-2 (VRSA) | 32 | 4 | 8 |
| VRS-4 (VRSA) | 32 | 4 | 16 |
| NRS-1 (VRSA) | 8 | 4 | 4 |
| 1593 (VRE) | >64 | 8 | 32 |
| 1751 (VRE) | >64 | 4 | 16 |
| 2431 (VRE) | >64 | 8 | 32 |
| 3183 (VRE) | >64 | 4 | 16 |
| 3222 (VRE | >64 | 8 | 16 |
| vanA VRE 700221 | 512 | 8 | — |
| vanB VRE 51299 | 256 | 2 | — |
| vanC-1 VRE 49608 | 32 | 0.5 | — |

| | | MIC (μg/ml) | MIC (μg/ml) |
|---|---|---|---|
| | MIC (μg/ml) | Conjugate 8 | Conjugate 4 |
| Organism ID | Vancomycin | (AV-0267) | (AV-0273) |
| ATCC 29213 MRSA | 0.5 | 1 | 2 |
| AR-978 MRSA | 1 | 1 | 1 |
| AR-979 MRSA | 1 | 0.5 | 1 |
| AR-993 MRSA | 1 | 1 | 1 |
| AR-997 MRSA | 1 | 1 | 1 |
| 6215 MRSA | 1 | 1 | 1 |
| 6231 MRSA | 0.5 | 0.5 | 1 |
| 6217 MRSA | 1 | 0.5 | 1 |
| 6218 MRSA | 1 | 1 | 1 |
| 6220 MRSA | 0.5 | 1 | 1 |
| VGP16-94 MSSA | 0.5 | 0.5 | 1 |
| VGP16-95 MSSA | 0.5 | 1 | 2 |
| VGP16-96 MSSA | 0.5 | 0.5 | 1 |
| VGP16-97 MSSA | 0.5 | 0.5 | 1 |
| VGP16-98 MSSA | 0.5 | 0.5 | 1 |
| VGP16-99 MSSA | 1 | 1 | 1 |
| VGP16-100 MSSA | 0.5 | 1 | 1 |
| VGP16-101 MSSA | 1 | 1 | 1 |
| VGP16-116 MSSA | 0.5 | 1 | 1 |
| VGP16-117 MSSA | 0.5 | 1 | 1 |
| ATCC 29212 E. faecalis | 2 | 1 | 2 |
| 1593 VRE | >64 | 32 | 8 |
| ATCC 49619 S. pneumoniae | 0.25 | 0.25 | 0.25 |
| MD77773 S. pneumoniae | 0.5 | 0.25 | 0.5 |
| CO314937 S. pneumoniae | 0.25 | 0.25 | 0.5 |
| 2008-1732 Group A Strep | 0.25 | ≤0.125 | 0.25 |
| 2008-818 Group A Strep | 0.25 | ≤0.125 | 0.25 |
| 2009-37 Group A Strep | 0.25 | ≤0.125 | 0.5 |
| 2008-87 Group B Strep | 0.5 | 0.25 | 0.5 |
| 2009-61 Group B Strep | 0.5 | 0.25 | 0.5 |
| 2009-62 Group B Strep | 0.5 | ≤0.125 | 0.25 |
| ATCC 25922 E. coli | >64 | >64 | >64 |
| AR-451 E. coli | >64 | >64 | >64 |
| AR-137 E. coli | >64 | >64 | >64 |

*Streptococci tested in CA-MHB w/5% LHB

| | | MIC (μg/ml) | MIC (μg/ml) |
|---|---|---|---|
| | MIC (μg/ml) | Conjugate 8 | Conjugate 4 |
| Organism ID | Vancomycin | (AV-0267) | (AV-0273) |
| ATCC 29213 MRSA | 0.5 | 1 | 1 |
| VRS-1 VRSA | >64 | 32 | 16 |
| VRS-2 VRSA | 32 | 8 | 4 |
| VRS-3a VRSA | 32 | 4 | 4 |
| VRS-3b VRSA | >64 | >64 | >64 |
| VRS-4 VRSA | >64 | 16 | 8 |
| VRS-5 VRSA | >64 | 32 | 8 |
| VRS-6 VRSA | >64 | 32 | 16 |
| VRS-7 VRSA | >64 | 32 | 8 |
| VRS-8 VRSA | >64 | 64 | 16 |
| VRS-9 VRSA | >64 | 64 | 16 |
| VRS-10 VRSA | >64 | 32 | 16 |
| VRS-11a VRSA | >64 | 64 | 16 |
| VRS-11b VRSA | >64 | 64 | 16 |
| NRS-1 VRSA | 8 | 4 | 4 |
| NRS-2 VRSA | 1 | 2 | 2 |

| | | MIC (μg/ml) | MIC (μg/ml) |
|---|---|---|---|
| | MIC (μg/ml) | Conjugate 8 | Conjugate 4 |
| Organism ID | Vancomycin | (AV-0267) | (AV-0273) |
| ATCC 29212 E. faecalis | 2 | 4 | 2 |
| 2431 VRE | >64 | 32 | 8 |
| 2059 VRE | >64 | 32 | 8 |
| 2574 VRE | >64 | 32 | 8 |
| 3053 VRE | >64 | 32 | 16 |
| 3183 VRE | >64 | 16 | 4 |
| 3190 VRE | >64 | 32 | 8 |
| 3191 VRE | >64 | 32 | 8 |
| 3222 VRE | >64 | 16 | 8 |
| 3235 VRE | >64 | 16 | 8 |
| 3239 VRE | >64 | 32 | 8 |
| 3241 VRE | >64 | 32 | 8 |
| 3623 VRE | >64 | 32 | 8 |
| 1751 VRE | >64 | 16 | 4 |
| 1772 VRE | >64 | 32 | 8 |
| 1946 VRE | >64 | 32 | 8 |

| | | MIC (μg/ml) | MIC (μg/ml) |
|---|---|---|---|
| | MIC (μg/ml) | Conjugate 8 | Conjugate 4 |
| Organism ID | Vancomycin | (AV-0267) | (AV-0273) |
| ATCC 49619 S. pneumoniae | ≤0.125 | 0.25 | 0.5 |
| CT0200-2241 S. pneumoniae | 0.25 | 0.25 | 0.5 |
| CT0200-2730 S. pneumoniae | 0.25 | 0.5 | 0.5 |
| CT3-5008 S. pneumoniae | 0.25 | 0.25 | 0.5 |
| CT3-5803 S. pneumoniae | 0.25 | 0.25 | 0.5 |
| CT3-8501 S. pneumoniae | 0.25 | 0.25 | 0.5 |
| CT4-3048 S. pneumoniae | 0.5 | 0.25 | 0.5 |
| CT4-9693 S. pneumoniae | 0.25 | 0.25 | 0.5 |
| CT5-6541 S. pneumoniae | 0.25 | 0.25 | 0.5 |
| CT5-8032 S. pneumoniae | 0.5 | 0.25 | 0.5 |
| CT6-0670 S. pneumoniae | 0.5 | 0.25 | 0.5 |
| CT5-9124 S. pneumoniae | 0.25 | 0.25 | 0.25 |
| CT6-7691 S. pneumoniae | 0.25 | 0.25 | 0.5 |
| CT4-9046 S. pneumoniae | 0.25 | ≤0.125 | 0.25 |

| | | MIC (μg/ml) | MIC (μg/ml) |
|---|---|---|---|
| | MIC (μg/ml) | Conjugate 8 | Conjugate 4 |
| Organism ID | Vancomycin | (AV-0267) | (AV-0273) |
| 2009-8 Group A Strep | 0.25 | 0.25 | 0.5 |
| 2008-45 Group A Strep | 0.25 | ≤0.125 | 0.5 |
| 2008-06 Group A Strep | 0.25 | ≤0.125 | 0.5 |
| 2007-107 Group A Strep | 0.25 | 0.25 | 0.5 |
| 2007-73 Group A Strep | 0.25 | ≤0.125 | 0.5 |
| 2007-49 Group A Strep | 0.25 | ≤0.125 | 0.5 |
| 2007-28 Group A Strep | 0.25 | <0.125 | 0.5 |
| 2007-11 Group A Strep | 0.25 | 0.25 | 0.5 |
| 2008-1692 Group A Strep | 0.25 | 0.25 | 0.5 |
| 2008-1565 Group A Strep | 0.25 | 0.25 | 0.5 |
| 2007-1101 Group A Strep | 0.25 | ≤0.125 | 0.5 |
| 2009-106 Group A Strep | 0.25 | 0.25 | 0.5 |
| 2009-109 Group A Strep | 0.25 | ≤0.125 | 0.5 |
| 2009-114 Group A Strep | 0.25 | 0.25 | 0.5 |
| 2009-136 Group A Strep | 0.25 | ≤0.125 | 0.5 |

| | | MIC (μg/ml) | MIC (μg/ml) |
|---|---|---|---|
| | MIC (μg/ml) | Conjugate 8 | Conjugate 4 |
| Organism ID | Vancomycin | (AV-0267) | (AV-0273) |
| 2009-60 Group B Strep | 0.5 | 0.25 | 0.5 |
| 2009-59 Group B Strep | 0.5 | 0.25 | 0.5 |
| 2009-57 Group B Strep | 0.5 | 0.25 | 0.5 |
| 2009-51 Group B Strep | 0.5 | ≤0.125 | 0.5 |
| 2009-43 Group B Strep | 0.5 | ≤0.125 | 0.5 |
| 2009-40 Group B Strep | 0.5 | 0.25 | 0.5 |
| 2009-20 Group B Strep | 0.5 | 0.25 | 0.5 |
| 2008-115 Group B Strep | 0.5 | 0.25 | 0.5 |
| 2008-57 Group B Strep | 0.5 | 0.25 | 0.5 |
| 2008-70 Group B Strep | 0.5 | ≤0.125 | 0.5 |
| J4222 Group B Strep | 0.5 | 0.25 | 0.5 |
| J4186 Group B Strep | 0.5 | 0.25 | 0.5 |
| J4185 Group B Strep | 0.5 | 0.25 | 0.5 |
| J4183 Group B Strep | 0.5 | 0.25 | 0.5 |
| J4182 Group B Strep | 0.25 | ≤0.125 | 0.5 |

In Vivo Efficacy Assays using a VRSA Thigh Infection Model

Example vancomycin-2,3-pyrrolidinediones conjugates were evaluated using an in vivo tissue infection model with vancomycin resistant MRSA:

Animal model: Murine thigh infection

Bacteria: VRSA (VRS-2). Gram Positive, Staphylococcus aureus (VRSA, VRS-2), Mouse Infected Thigh Model. VRS-2 is VanA producing SCC Mec II st5 train.

Antibiotic resistance profile: vancomycin (>64 μg/mL), quinolones, macrolides and trimethoprim-sulfamethoxazole.

Target density: 1*10⁵ CFU/mouse Actual count of bacteria: 8.4*10⁴ CFU/mouse

Vancomycin conjugates dose: 100 mg/Kg intraperitoneally (IP).

Application: This model assesses the antimicrobial efficacy of test articles in a tissue infection model with vancomycin resistant MRSA. Microbial counts are measured.

Organism: S. aureus strain VRS-2, VRSA strain Hershey, is a Van-A producing SCC Mec II, st5 strain that was isolated from the foot ulcer of a 70-year-old patient. It is methicillin-resistant with resistance to carbapenems, cephalosporins and penicillins. VRS-2 is resistant to vancomycin (MIC>64), quinolones (LVX-R, CIP—R), macrolides (ERY-R, CLI-R), and trimethoprim sulfamethoxazole.

Procedure: Groups of 5 female specific-pathogen-free ICR mice were used. Animals are immunosuppressed by two intraperitoneal injections of cyclophosphamide, the first at 150 mg/kg 4 days before infection (day −4) and the second at 100 mg/kg 1 day before infection (day −1). On day 0, animals are inoculated intramuscularly (0.1 ml/thigh) with Staphylococcus aureus, vancomycin resistant (VRS-2) into the right thigh. Vehicle and/or test substances are then administered per the study design table instructions. At 24 hours after treatment initiation, animals are humanely euthanized with CO₂ asphyxiation then the muscle of the right thigh is harvested from each test animal. The removed muscle tissues are homogenized in 5 ml of PBS, pH 7.4, with a polytron homogenizer. Homogenates, 0.1 ml, are used for serial 10-fold dilutions and plated on nutrient agar plates for colony count (CFU/g) determination.

Reference Data: Linezolid—50, BID PO
Study Design and Protocol (Staphylooccus aureus (VRSA, VRS-2), Mouse Infected Thigh Model)

| | | Dose specifications | | | | | | |
|---|---|---|---|---|---|---|---|---|
| | | | | | | | Total | |
| | | | | | | Each | admin | |
| | Test | | | Conc. | Vol | admin | (mg/kg/ | Mice |
| Group | article | Route | Schedule | (mg/mL) | (mL/kg) | (mg/kg) | day) | (female) |
| 1 | Untreated | — | — | — | — | — | — | 5 |
| 2 | Vehicle | IP | BID × 1 | — | 12.5 | — | — | 5 |
| | | | day | | | | | |
| 3 | Linezolid | PO | BID × 1 | 5 | 10 | 50 | 100 | 5 |
| | reference | | day | | | | | |
| | standard | | | | | | | |
| 4 | TA-1 AV- | IV | BID × 1 | 20 | 5 | 100 | 200 | 5 |
| | 0273 | | day | | | | | |
| 5 | TA-2 AV- | IV | BID × 1 | 20 | 5 | 100 | 200 | 5 |
| | 0267 | | day | | | | | |

Target inoculum: 10⁵ CFU/mouse.

Group 1. Untreated control. Animals will be sacrificed at 2 h after inoculation for initial colony counts.

Groups 2, 4-5. The test articles and vehicle will be administered intravenously (IV) or intraperitoneally (IP) twice (BID) with a 12 h interval (q12h) at 2 h and 14 h after infection.

Group 3. The reference standard, linezolid, will be administered orally (PO) twice (BID) with a 12 h interval (q12h) at 2 h and 14 h after infection.

Animals were sacrificed at 26 h after infection. Colony counts will be determined from infected thigh tissue.

Animals were observed for symptoms of acute toxicity at 5 min after IV dosing or 30 min after IP dosing.

The strain S. aureus VRS-2 grew well and resulted in greater than a 3-log₁₀ increase in bacterial counts over the 24 h treatment period. The baseline count at 2h post-infection were 5.34-log₁₀ CFU/g thigh, corresponding to 5.28-log₁₀ CFU/thigh. The bacterial counts in the vehicle control group at 26 h post-infection were 8.44-log₁₀ CFU/g thigh, corresponding to 8.38-log₁₀ CFU/thigh. The reference control agent, linezolid at 50 mg/kg PO BID q12h, yielded a significant reduction (100% reduction, p<0.05) in bacterial counts compared to the vehicle control group.

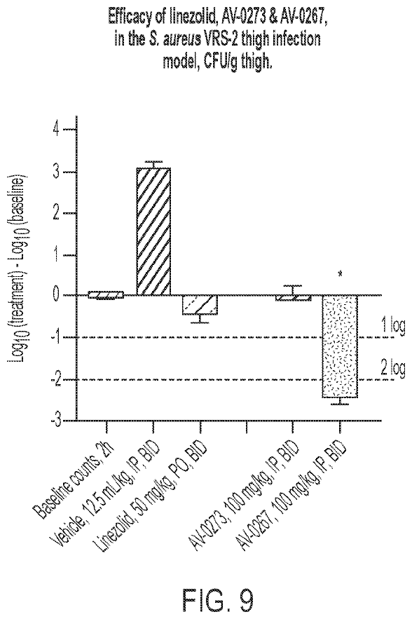

The IP administration of Compound 8 at 100 mg/kg BID q12h resulted in a significant bacterial count reduction (100% reduction) in the thigh tissue relative to the vehicle control group (p<0.05), as well as a significant 2.51-log₁₀ (CFU/g thigh) or 2.46-log₁₀ reduction (CFU/thigh) relative to the baseline control was observed (p<0.05, FIG. 9).

The IP administration of Compound 4 at 100 mg/kg BID q12h resulted in a significant bacterial count reduction (100% reduction) in the thigh tissue relative to the vehicle control group(p<0.05), as well as a bacteriostatic effect was found relative to the baseline control (FIG. 9).

The compounds, compositions, and methods of the appended claims are not limited in scope by the specific compounds, compositions, and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, compositions, and method steps disclosed herein are specifically described, other combinations of the compounds, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A compound defined by Formula I 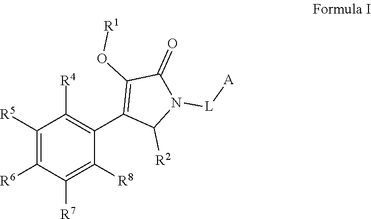
or a pharmaceutically acceptable salt or prodrug thereof, wherein
L is absent, or represents a bivalent linking group;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl.

2. The compound of any of the preceding claims, wherein R¹ is hydrogen or a C₁-C₄ alkyl group optionally substituted with one or more substituents individually chosen from R⁹.

3. The compound of any of the preceding claims, wherein R² is a C₁-C₄ alkyl group optionally substituted with one or more substituents individually chosen from R⁹.

4. The compound of any of claims 1-3, wherein at least one of R⁴, R⁵, R⁶, R⁷, and R⁸ is not hydrogen.

7. The compound of any of claims 1-3, wherein R⁴, R⁵, R⁷, and R⁸ are hydrogen.

8. The compound of any of the preceding claims, wherein R⁶ is an electron withdrawing group.

9. The compound of any of the preceding claims, wherein R⁶ is haloalkyl.

10. The compound of any of the preceding claims, wherein R⁶ is perfluoroalkyl.

11. The compound of any of the preceding claims, wherein R⁶ is —CF₃.

12. The compound of any of the preceding claims, wherein A comprises an antibacterial agent.

13. The compound of claim 12, wherein the antibacterial agent acts via an extracellular mechanism of action.

14. The compound of claim 13, wherein the antibacterial agent targets bacterial cell walls.

15. The compound of any of claims 12-14, wherein the antibacterial agent comprises a β-lactam, such as a penicillin, a cephalosporin, a monobactam, or a carbapenem.

16. The compound of any of claims 12-14, wherein the antibacterial agent comprises a glycopeptide, such as teicoplanin, vancomycin, telavancin, dalbavancin, or oritavancin.

17. The compound of any of claims 12-14, wherein the antibacterial agent comprises a polypeptide antibiotic, such as bacitracin.

18. The compound of any of the preceding claims, wherein L comprises from 2 to 30 carbon atoms.

19. The compound of any of the preceding claims, wherein L comprises an alkylene group, a cycloalkylene group, an alkylcycloalkylene group, an arylene group, an alkylarylene group, an oligo(alkyleneoxy) group, an oligo(alkyleneimine) group, or any combination thereof.

20. The compound of 19, wherein L further comprises one or more functional groups, such as a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—), wherein R⁹ represents an alkyl group, an aryl group, or a heterocyclic group.

21. The compound of any of the preceding claims, wherein L is not cleavable.

22. The compound of any of the preceding claims, wherein L comprises a positively charged moiety.

23. The compound of any of claims 1-22, wherein the compound is defined by Formula IA 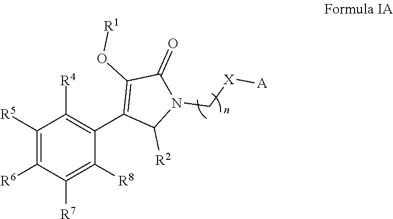
or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
n is an integer from 2 to 12;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

24. The compound of any of claims 1-22, wherein the compound is defined by Formula IB 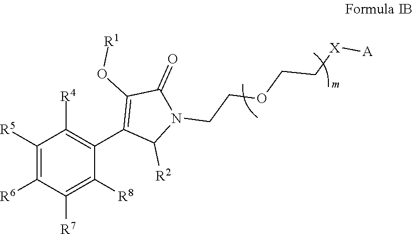
or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
m is an integer from 1 to 20;
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

25. The compound of any of claims 1-22, wherein the compound is defined by Formula IC or Formula ID 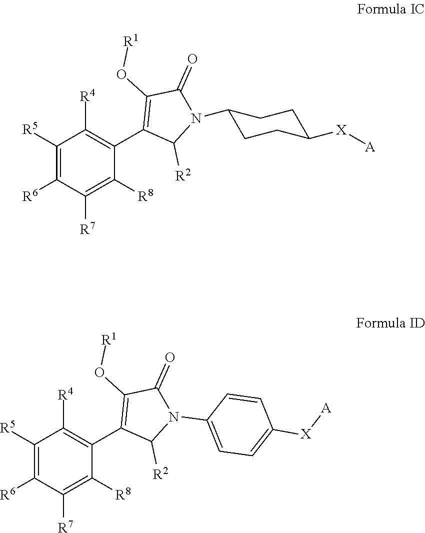
or a pharmaceutically acceptable salt or prodrug thereof, wherein
X is absent, or comprises a functional groups chosen from a secondary amine (—NH—), a tertiary amine (—NR⁹—), a secondary amide (—CONH—), tertiary amide (—CONR⁹—), secondary carbamate (—OCONH—; —NHCOO—), tertiary carbamate (—OCONR⁹—; —NR⁹COO—), urea (—NHCONH—; —NR⁹CONH—; —NHCONR⁹—, or —NR⁹CONR⁹—), carbinol (—CHOH—, —CR⁹OH—), ether (—O—), or ester (—COO—, —CH₂O₂C—, CHR⁹O₂C—);
A comprises an antimicrobial agent;
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and
R⁹ is hydrogen, alkyl, alkenyl, alkynyl, aryl, heteroaryl, cycloalkyl, or heterocycloalkyl.

26. A composition comprising a compound defined by any of claims 1-25 in a pharmaceutically acceptable carrier.

27. A biofilm preventing, removing, or inhibiting composition comprising a carrier and an effective amount of a compound defined by any of claims 1-25.

28. The composition of claim 27, wherein the composition is a dentifrice composition that promotes dental hygiene by preventing, reducing, inhibiting or removing a biofilm.

29. The composition of claim 28, wherein the dentifrice composition comprises a toothpaste, mouthwash, chewing gum, dental floss, or dental cream.

30. A method of controlling biofilm formation on a substrate comprising contacting the substrate with a compound defined by any of claims 1-25.

31. The method of any of claims 27-30, wherein the biofilm comprises Gram-positive bacteria.

32. The method of claim 31, wherein the biofilm comprises bacteria of a genus Staphylococcus.

33. The method of claim 32, wherein the biofilm comprises bacteria of the species Staphylococcus aureus.

34. A method for treating a chronic bacterial infection in a subject in need thereof, comprising administering to said subject a compound defined by any of claims 1-25.

35. The method of claim 34, wherein the chronic bacterial infection is chosen from urinary tract infection, gastritis, respiratory infection, cystitis, pyelonephritis, osteomyelitis, bacteremia, skin infection, rosacea, acne, chronic wound infection, infectious kidney stones, bacterial endocarditis, and sinus infection.

36. A medical device comprising:
(a) a medical device substrate; and
(b) an effective amount of a compound defined any of claims 1-25.

37. The medical device of claim 36, wherein the medical device substrate is chosen from stents, fasteners, ports, catheters, scaffolds and grafts.

38. A method of treating a subject infected with a bacterium comprising administering to the subject a therapeutically effective amount of a compound defined by any of claims 1-25

39. The method of claim 38, wherein the bacterium comprises a Gram-positive bacterium.

40. The method of claim 39, wherein the bacterium is chosen from Staphylococcus aureus (methicillin sensitive), Staphylococcus aureus (methicillin resistant), Staphylococcus aureus (vancomycin resistant), Streptococcus pneumonia (penicillin sensitive), Streptococcus pneumonia (penicillin resistant), Staphylococcus epidermis (multiple drug resistant), Enterococcus faecalis (vancomycin sensitive), Enterococcus faecium (vancomycin resistant), and Haemophilus influenzae.

41. A method of overcoming acquired resistance to an antimicrobial agent, the method comprising conjugating the antimicrobial agent to a 2,3-pyrrolidinedione defined by the structure below: 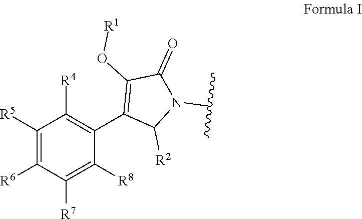
or a pharmaceutically acceptable salt or prodrug thereof, wherein
R¹ is chosen from hydrogen, alkyl, haloalkyl, alkenyl, haloalkenyl, alkynyl, and haloalkynyl, each optionally substituted with one or more substituents individually chosen from R³;
R² is chosen from hydrogen, halogen, alkyl, haloalkyl, alkylthio, haloalkylthio, alkoxy, haloalkoxy, alkenyl, haloalkenyl, alkynyl, haloalkynyl, cycloalkyl, hetercycloalkyl, alkylcycloalkyl, alkylhetercycloalkyl, aryl, heteroaryl, alkylaryl, and alkylheteroaryl, each optionally substituted with one or more substituents individually chosen from R³;
R³ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl; and R⁴, R⁵, R⁶, R⁷, and R⁸ are each independently chosen from hydrogen, halogen, hydroxyl, —CN, —NO₂, amino, alkylamino, dialkylamino, alkyl, haloalkyl, alkylthio, haloalkylthio, alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl.
